(19) United States
(12) Patent Application Publication
(10) Pub. No.: US 2013/0012706 A1
(43) Pub. Date: Jan. 10, 2013

(54) Methods and Intermediates for the Synthesis of 4-oxo-3,4-dihydro-imidazo[5,1-d][1,2,3,5]tetrazines

(75) Inventors: Marc Geoffery Hummersone, Nottingham (GB); David Cousin, Nottingham (GB)

(73) Assignee: PHARMINOX LIMITED, Nottingham (GB)

(21) Appl. No.: 13/581,874

(22) PCT Filed: Feb. 25, 2011
(86) PCT No.: PCT/GB2011/000266

§ 371 Date: Aug. 30, 2012

Related U.S. Application Data
(60) Provisional application No. 61/309,111, filed on Mar. 1, 2010.

(51) Int. Cl. C07D 487/04

(52) U.S. Cl. 544/179

(57) ABSTRACT

The present invention provides a compound of general formula (II), or a salt or solvate thereof:

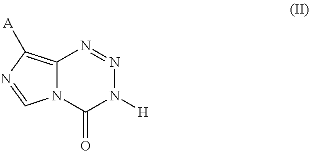

wherein
A is independently -A¹, -A², -A³, -A⁴, -A⁵, -A⁶, or -A⁷, wherein:
   -A¹ is independently C₅₋₁₂heteroaryl, and is optionally substituted;
   -A² is independently thioamido or substituted thioamido;
   -A³ is independently imidamido, substituted imidamido, N-hydroxyimidamido, or substituted N-hydroxyimidamido;
   -A⁴ is independently hydroxamic acid or hydroxamate;
   -A⁵ is independently carboxamide or substituted carboxamide;
   -A⁶ is independently aliphatic C₂₋₆alkenyl, and is optionally substituted; and
   -A⁷ is independently carboxy or C₁₋₄alkyl-carboxylate;
      and its use in the synthesis of temozolomide and analogues thereof.

### TECHNICAL FIELD

This invention pertains generally to processes, methods and materials for the preparation of imidazo[5,1-d][1,2,3,5]tetrazine compounds, such as temozolomide and its analogues and, in particular, to certain chemical intermediates for use in said processes. The imidazo[5,1-d][1,2,3,5]tetrazine compounds are useful as drugs, for example, in the treatment of tumours.

### BACKGROUND

A number of patents and publications are cited herein in order to more fully describe and disclose the invention and the state of the art to which the invention pertains. Each of these references is incorporated herein by reference in its entirety into the present disclosure, to the same extent as if each individual reference was specifically and individually indicated to be incorporated by reference.

Throughout this specification, including the claims which follow, unless the context requires otherwise, the word “comprise,” and variations such as “comprises” and “comprising,” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Thus, for example, reference to “a pharmaceutical carrier” includes mixtures of two or more such carriers, and the like.

Ranges are often expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

### Temozolomide and Analogues

Temozolomide (also known as 3,4-dihydro-3-methyl-4-oxoimidazo[5,1-d]-1,2,3,5-tetrazine-8-carboxamide; 8-carbamoyl-3-methylimidazo[5,1-d]-1,2,3,5-tetrazin-4(3H)-one; methazolastone; M & B 39831; CCRG-81045; NSC-362856; Temodal; Temodar) is a well known anti-neoplastic agent that acts as an alkylating agent. Its primary application is in the treatment of brain cancer (e.g., glioma).

Temozolomide has the structure shown below, in which the ring atoms are numbered for ease of reference:

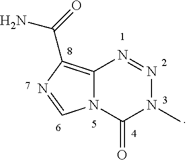

Temozolomide is the subject of granted claim 13 of U.S. Pat. No. 5,260,291 to Lunt et al. granted 9 Nov. 1993.

Certain analogues of temozolomide, which are 3-substituted-4-oxo-3,4-dihydroimidazo[5,1-d][1,2,3,5]tetrazine-8-carboxylic acid amides, are described in international patent application number PCT/GB2008/004140 filed 16 Dec. 2008 (published as WO2009/077741 on 25 Jun. 2009), which is herein incorporated by reference.

Further analogues of temozolomide, which are 3-substituted-8-substituted-3H-imidazo[5,1-d][1,2,3,5]tetrazine-4-ones have been described in co-pending U.S. application 61/219,575, filed 23 Jun. 2009, which is herein incorporated by reference.

Analogues of temozolomide have been shown to have good activity against tumour cell lines.

### Synthesis of Temozolomide and Analogues

Several methods for the chemical synthesis of temozolomide and its analogues have been described.

In one approach (see, for example, Stevens et al, J. Med. Chem., 1984, 27, 196-201) a suitable isocyanate is reacted with 5-diazoimidazole-4-carboxamide to give the corresponding 3-substituted imidazotetrazine, as illustrated in the following scheme.

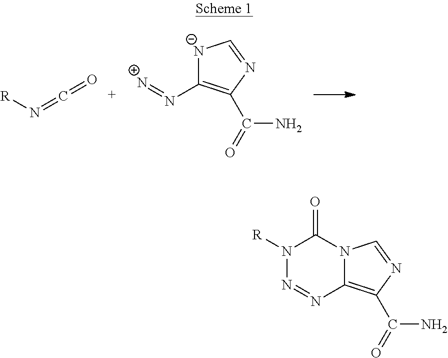

To obtain temozolomide itself, this synthetic route involves the use of the reagent methyl isocyanate, which is particularly toxic and volatile and is therefore a very difficult and dangerous chemical to work with. Methyl isocyanate was the main chemical involved in the Bhopal disaster in which over half a million people were exposed to toxic emissions from a pesticide plant, resulting in thousands of deaths.

Kuo et al suggested a slightly different method (see, for example U.S. Pat. No. 7,087,751), as shown in the scheme below.

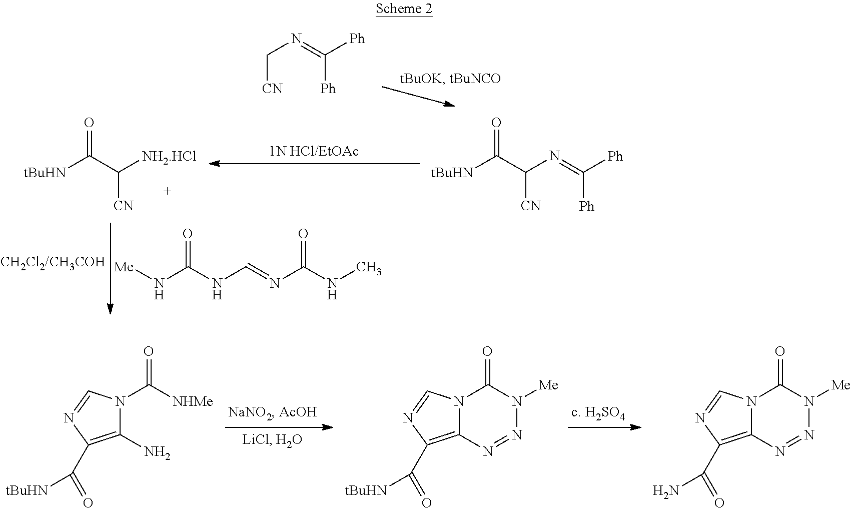

Wanner et al (J. Chem. Soc., Perkin Trans. I, 2002, 1877-1880) also disclosed an alternative method for the synthesis of temozolomide, based on a condensation reaction between a nitrosoimidazole and phenylmethylcarbazate as shown in the following scheme.

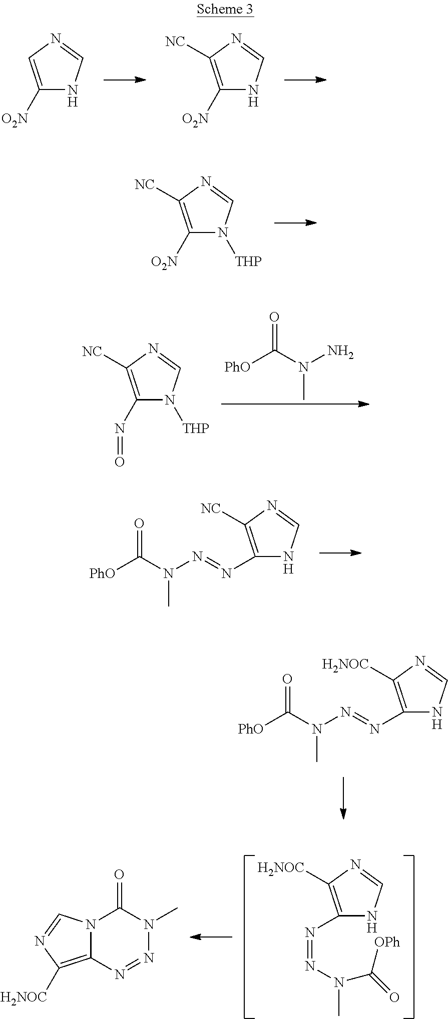

Although this route does not use methyl isocyanate, it is rather a long route, involving many more steps compared to the method of Stevens et al.

In another approach, as discussed in WO2009/077741, for example, the 3-(hydroxymethyl) compound (3-hydroxymethyl-4-oxo-3,4-dihydro-imidazo[5,1-d][1,2,3,5]tetrazine-8-carboxylic acid amide) is used as an intermediate. This intermediate may be prepared by methods such as in the following scheme.

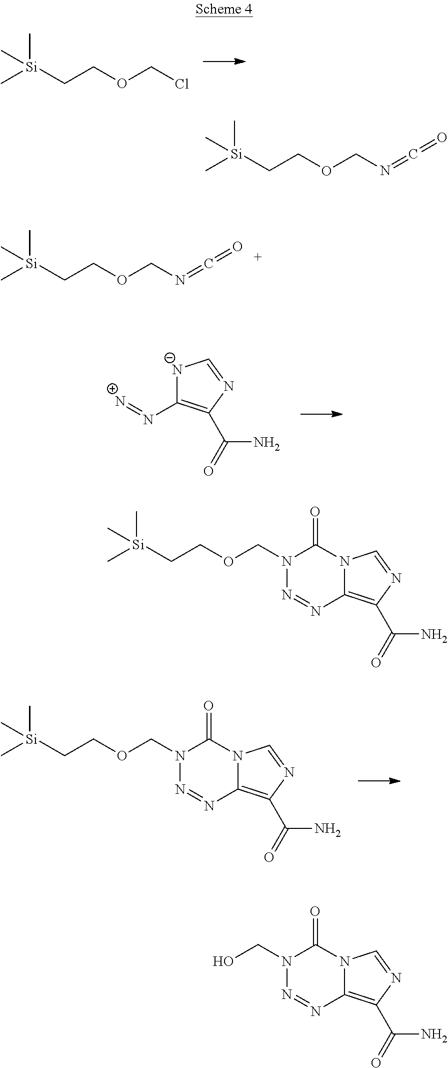

This intermediate may then be used to prepare a range of other 3-substituted compounds e.g. by reaction with a suitable alkyl halide or electrophile.

There is a need for an alternative method of synthesis of temozolomide and analogous compounds, which avoids the need to use highly toxic materials such as methyl isocyanate. There is also a need for an alternative process that can be reproduced on a large scale and/or provide increased yields of product in a high degree of purity, preferably reducing or removing the need for additional purification and isolation steps.

The present inventors have established a new route, via a key intermediate, that avoids the use of methyl isocyanate and which can produce high purity temozolomide in good yield via a robust, scalable process. The new process may also have other advantages in terms of environmental benefits and reduced cost of materials.

### SUMMARY OF THE INVENTION

The present inventors have now identified new methods for the synthesis of temozolomide and its analogues. New intermediate compounds, for use in the synthesis of temozolomide and its analogues have also been identified.

In one aspect, therefore, the invention provides a compound of general formula (II) as defined herein, or a salt or solvate thereof.

Also provided are methods for the synthesis of compounds of formula (II).

In another aspect, the invention provides the use of a compound of general formula (II) as defined herein in the synthesis of temozolomide or an analogue thereof.

In another aspect, the invention provides the use of a compound of general formula (II) as defined herein in the synthesis of a compound of formula (I) as defined herein.

In another aspect the present invention provides a method for the synthesis of temozolomide or an analogue thereof comprising the step of alkylating a compound of formula (II).

In another aspect the present invention provides a method for the synthesis of a compound of formula (I) as defined herein, comprising the step of alkylating a compound of formula (II).

In another aspect, the invention provides compounds of formula Op as defined herein and their use in the synthesis of compounds of formulae (II) and (I).

### DETAILED DESCRIPTION OF THE INVENTION

The present invention relates to compounds of formula (II)

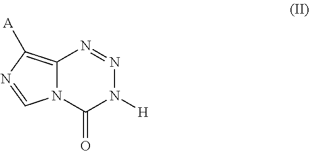

wherein:
A is independently selected from -A¹, -A², -A³, -A⁴, -A⁵, -A⁶, -A⁷, -A⁸, -A⁹, -A¹⁰ and -A¹¹ wherein:
-A¹ is independently C₅₋₁₂heteroaryl, and is optionally substituted;
-A² is independently thioamido or substituted thioamido;
-A³ is independently imidamido or substituted imidamido;
-A⁴ is independently hydroxamic acid or hydroxamate;
-A⁵ is independently carboxamide or substituted carboxamide;
-A⁶ is independently aliphatic C₂₋₆alkenyl, and is optionally substituted; and
-A⁷ is independently carboxy or C₁₋₄alkyl-carboxylate.

The invention also relates to compounds of formula (III):

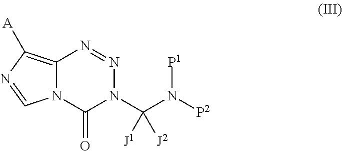

wherein A is as defined above;
J¹ and J² are each independently H or C₁₋₃ alkyl; and
P¹ and P² are each independently H or an amine protecting group or P¹ and P² together form an amine protecting group.

The present invention also relates to a method for the synthesis of compounds of formula (II) from compounds of formula (III).

The present invention also relates to a method for the synthesis of temozolomide or a temozolomide analogue, for example a compound of formula (I):

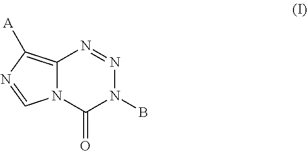

wherein A is as defined above and B is independently -B¹, -B², -B³, -B⁴, -B⁵, -B⁸, -B⁷, -B⁸, -B⁹, -B¹⁰, -B¹¹, B¹², -B¹³, -B¹⁴, -B¹⁵, or -B¹⁶;
wherein:
-B¹ is independently saturated aliphatic C₁₋₆alkyl;
-B² is independently aliphatic C₂₋₆alkynyl;
-B³ is independently mercapto-C₁₋₄alkyl, sulfanyl-C₁₋₄alkyl, sulfinyl-C₁₋₄alkyl, or sulfonyl-C₁₋₄alkyl;
-B⁴ is independently hydroxy-C₁₋₄alkyl or ether-C₁₋₄alkyl;
-B⁵ is independently phenyl-C₁₋₆alkyl or C₅₋₁₂heteroaryl-C₁₋₆alkyl, and is optionally substituted;
-B⁶ is independently acyl-C₁₋₆alkyl, carboxy-C₁₋₆alkyl, oxyacyl-C₁₋₆alkyl, or acyloxy-C₁₋₆alkyl;
-B⁷ is independently amido-C₁₋₆alkyl or substituted amido-C₁₋₄alkyl;
-B⁸ is independently C₃₋₆cycloalkyl, C₃₋₆cycloalkyl-C₁₋₄alkyl, C₃₋₆heterocyclyl, or C₃₋₆heterocyclyl-C₁₋₄alkyl, and is optionally substituted;
-B⁹ is independently halo-C₁₋₆alkyl;
-B¹⁰ is independently nitro-C₁₋₆alkyl;
-B¹¹ is independently cyano-C₁₋₆alkyl;
-B¹² is independently phosphate-C₁₋₆alkyl;
-B¹³ is independently carbamate-C₁₋₆alkyl; and
-B¹⁴ is independently oxime-C₁₋₆alkyl.
   comprising reacting a compound of formula (II), as defined above, with a suitable electrophile.

### Compounds of Formula (II) and (III)

The present inventors have developed an improved route to temozolomide and related compounds, which proceeds via an intermediate of general formula (II)

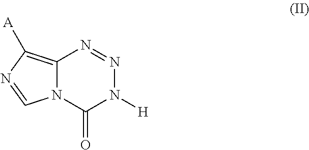

(wherein group A is the 8-substituent, and is as defined herein).

The intermediate of general formula (II) has a N—H group at the 3-position. It is thought that compounds of this type have not previously been synthesised. In particular, the inventors have been able to synthesise the key intermediate known as nortemozolomide, which has the structure below.

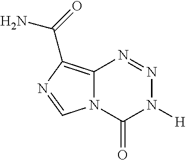

Brown et al (J. Med. Chem., 2002, 45, 25, 5448-5457) describe a failed attempt to synthesise nortemozolomide by demethylation of temozolomide.

Wang (Bioorg. Med. Chem. Lett., 1996, 6, 2, 185-188) disclose a compound claimed to be nortemozolomide and propose a synthetic route for its preparation.

However, Wang's attempts to methylate the product obtained from the proposed synthesis, in the hope of converting it to temozolomide, were unsuccessful, resulting in a complex mixture, which was not properly characterised.

In contrast, methylation of nortemozolomide produced by the method of the present invention proceeds, as predicted, to give temozolomide. This is shown in the present examples. This confirms that the compound produced by the present method, unlike that claimed by Wang at al, is indeed nortemozolomide and that it is a useful intermediate in the synthesis of temozolomide.

The analytical data for the compound produced by the method described in the Wang paper is inconsistent with the analytical data for nortemozolomide produced by the method of the present inventors. This can be seen from a comparison of the ¹H, ¹³C, and ¹⁵N NMR, IR and mass spectra of nortemozolomide, disclosed herein, with those published by Wang.

Therefore, it appears that the authors of that paper misinterpreted the product of their reactions. In other words, the product obtained by Wang was not actually nortemozolomide, but was some other compound.

It is also noted that Professor Malcolm Stevens was named as a co-author in the above-discussed paper without his knowledge or consent.

The present inventors have therefore provided, for the first time, an enabling disclosure of the synthesis of nortemozolomide and its conversion to temozolomide.

The method of the present invention is also applicable to the synthesis of other compounds of formulae (I) and (II).

Intermediate compounds of formula (II) can conveniently be converted to temozolomide and analogues thereof, for example to compounds of formula (I), by alkylation at the 3-NH group.

This provides a versatile route to temozolomide and related analogues with various groups in the 3-position.

Intermediate compounds of formula (II) may be produced from compounds of formula (III) as defined above, by deprotecting the 3-NH group.

Compounds of formula (III) may be synthesised, for example by reaction of an isocyanate of general formula (IV):

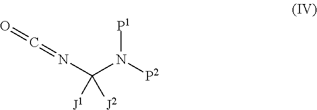

wherein J¹, J², P¹, P² are as defined herein
with a diazoimidazole compound of general formula (V):

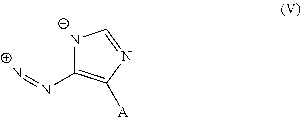

wherein A is as defined herein.

Isocyanates of formula (IV) may be prepared from an appropriate protected amino acid, of general formula (VI):

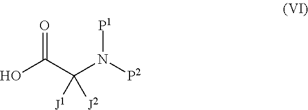

wherein J¹, J², P¹, P² are as defined herein.
Production of Compounds of Formula (II) from Compounds of Formula (III) Deprotection Step

Compounds of formula (II) can be prepared from compounds of formula (III):

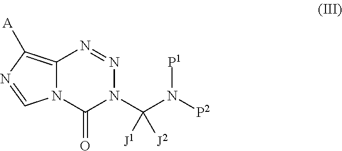

wherein A, J¹, J², P¹ and P² are as defined herein.

The step of deprotecting the compound of formula (III), to provide the corresponding 3-NH compound of formula (II) is referred to herein as the deprotection step. The inventors have found that the protected nitrogen eliminates spontaneously upon removal of P¹ and P² to give the free 3-NH.

The conditions needed for the deprotection step will depend on the nature of the amine protecting group(s) P¹ and/or P². Methods for the removal of amine protecting groups are known in the art. See, for example, Protective Groups in Organic Synthesis (T. Green and P. Wuts; 4th Edition; John Wiley and Sons, 2006).

In some embodiments, in the deprotection step, the compound of formula (III) is treated with acid.

In some embodiments, in the deprotection step, the compound of formula (III) is treated with aqueous hydrochloric acid.

In some embodiments, in the deprotection step, the acid is at a concentration of from 0.5N to 5N.

In some embodiments, in the deprotection step, the acid is at a concentration of about 3N.

In some embodiments, the deprotection step is carried out at room temperature or below.

In some embodiments, the deprotection step is carried out at room temperature.

In some embodiments, the compound of formula (II) may precipitate out of the reaction mixture.

In some embodiments, after reaction, the compound of formula (II) is isolated by filtration.

In some embodiments, after reaction, the compound of formula (II) is washed, for example with water and one or more organic solvents.

In some embodiments the organic solvents comprise ethyl acetate.

In some embodiments, the organic solvents comprise diethyl ether.

Advantageously, the compound of formula (II) may not require further purification.

### Synthesis of Compounds of Formula (III)

Compounds of formula (III) may be prepared by methods analogous to known methods for the synthesis of temozolomide and its analogues, wherein a suitable isocyanate is reacted with a diazoimidazole to give the corresponding 3-substituted imidazotetrazine (for example, as illustrated in Scheme 1, above).

See, for example, Wang, Y., et al., 1998, “Antitumour imidazotetrazines. Part 36. Conversion of 5-amino-imidazole-4-carboxamide to imidazo[5,1-d][1,2,3,5]tetrazin-4(3H)-ones and imidazo[1,5-a][1,3,5]triazin-4(3H)-ones related in structure to the antitumour agents temozolomide and mitozolomide,” J. Chem. Soc., Perkin Trans 1, Vol. 10, pp. 1669-1675; Stevens, M. F. G., et al., 1984, “Antitumour imidazotetrazines. Part 1. Synthesis and chemistry of 8-carbamoyl-3-(2-chloroethyl)imidazo[1,5-d]-1,2,3,5-tetrazin-4(3H)-one, a novel broad spectrum antitumour agent”, J. Med. Chem., Vol. 27, pp. 196-201.

Accordingly, compounds of formula (III) can be prepared by reaction of an isocyanate of general formula (IV):

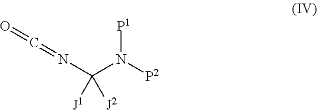

with a diazoimidazole compound of general formula (V):

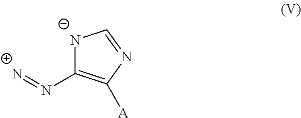

wherein A is as defined herein.

Suitable isocyanates may be obtained from commercial sources, or prepared using known methods, or by adapting known methods in known ways. For example, methods for preparing certain isocyanates are described in WO 96/27588.

The classical routes to isocyanates are treatment of a primary amine with phosgene, or a phosgene equivalent, and the Curtius rearrangement of an acyl azide (see, e.g., Ozaki, S., 1972, Chem. Rev., Vol. 72, pp. 457-496; Saunders, J. H., et al., 1948, Chem. Rev., Vol. 43, pp. 203-218). Acyl azides are commonly prepared by the treatment of an acid chloride with sodium azide or, more conveniently, are prepared directly from the carboxylic acid using diphenylphosphoryl azide (dppa) (see, e.g., Shioiri, T., et al., 1972, J. Am. Chem. Soc., Vol. 94, pp. 6203-6205) and are not normally isolated.

In some embodiments, the isocyanate of formula (IV) is tert-butyl isocyanatomethylcarbamate.

tert-Butyl isocyanatomethylcarbamate may be prepared from N-Boc-glycine and ethyl chloroformate, as shown in the scheme below:

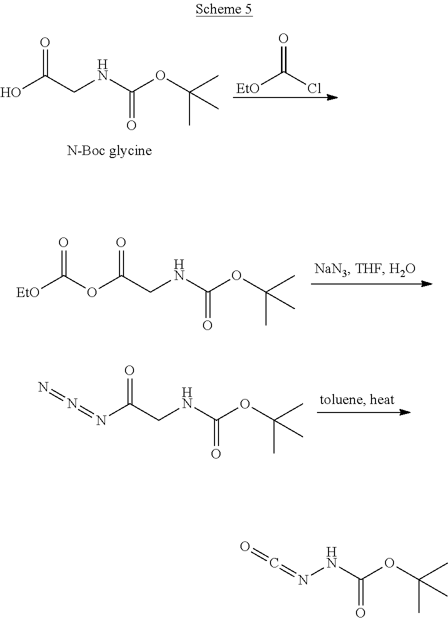

This method is adapted from that described in J. Chem. Soc., Perkin Trans. 1, 2000, 4328-4331.

Other compounds of formula (IV) may be prepared, in an analogous manner, from an appropriate protected amino acid, of general formula (VI):

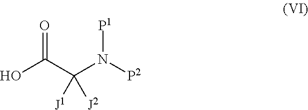

Compounds of formula (VI) may be obtained from commercial sources, or prepared using known methods, or by adapting known methods in known ways.

In some embodiments, the compound of formula (V) is 5-diazoimidazole-4-carboxamide.

5-Diazoimidazole-4-carboxamide is a known reagent. Other compounds of formula (V) could be prepared, for example by converting a carboxamide group to another group A as defined herein, as discussed above. In particular, a carboxamide group may be converted to a group of formula -A¹, -A², -A³, -A⁴, -A⁵, -A⁶, or -A⁷ as described herein. In some embodiments a 5-aminoimidazole-4-carboxamide may be derivatised to produce a group A, and the amino group then diazotised.

Other compounds of formula (VI) may be obtained from commercial sources, or prepared using known methods or by adapting known methods in known ways.

In some embodiments, the reaction of compound (IV) with compound (V) is performed under an inert atmosphere.

In some embodiments, the reaction of compound (IV) with compound (V) is performed under an argon or nitrogen atmosphere.

In some embodiments, the reaction is performed in an organic solvent.

In some embodiments, the organic solvent is DMSO.

In some embodiments, the compound of formula (III) may precipitate out of the reaction mixture.

In some embodiments, after reaction, the compound of formula (III) is isolated by filtration.

In some embodiments, after reaction, the compound of formula (III) is washed, for example with water and one or more organic solvents.

In some embodiments the organic solvents comprise ethyl acetate.

In some embodiments, the organic solvents comprise diethyl ether.

### Electrophile Addition Step:

Production of Compounds of Formula (I) from Compounds of Formula (II)

As will be appreciated by those skilled in the art, the 3-NH group of a compound of formula (II) can be reacted with an electrophile.

For example, the 3-NH group may be reacted with a suitable electrophile to produce various groups, for example N-alkyl and substituted N-alkyl groups, at the 3-position. For convenience this is referred to hereafter as the ‘electrophile addition step’. In some embodiments, it may be referred to as the alkylation step.

In some embodiments, the electrophile addition step is performed in a reaction medium comprising an organic solvent.

In some embodiments, the solvent is DMF (dimethylformamide).

In some embodiments, the reaction is performed in an aqueous medium.

In some embodiments the electrophile addition step is performed at room temperature.

In some embodiments the electrophile addition step is performed at a temperature less than room temperature.

In some embodiments, less than room temperature is 10° C. or less.

In some embodiments, less than room temperature is 5° C. or less.

In one embodiment, less than room temperature is 0° C. or less.

Suitable alkylating agents for use in the electrophile addition (alkylation) step are known in the art and include, but are not limited to, alkyl halides, epoxides, alkyl alcohols, activated alkyl alcohols (for example, an alkyl alcohol in the presence of triphenylphosphine), alkyl alkoxides.

Aldehydes may also be reacted with the 3-NH group, to produce hydroxy-substituted N-alkyl groups at the 3-position.

In some embodiments, the electrophile is an alkylating agent.

In some embodiments, the alkylating agent is an alkyl halide.

In some embodiments, the alkylating agent is a C₁₋₆ alkyl halide.

In some embodiments, the alkylating agent is a C₁₋₆ alkyl iodide.

In some embodiments, the alkylating agent is methyl iodide.

In some embodiments, the alkylating agent is a C₁₋₆ alkynyl halide.

In some embodiments, the alkylating agent is propargyl bromide.

In some embodiments, the alkylating agent used in the alkylation step may be a compound of formula B—X, where B is as defined herein for the compounds of formula (I), and X is a leaving group.

Suitable leaving groups are known in the art and include, but are not limited to, halide (—F, —Cl, —Br, —I), alkoxide (—OR), hydroxide (—OH), water (—⁺OH₂), alcohol (—⁺OHR), sulfonates such as tosylate (—OTs; —OSO₂(p-MePh)) or mesylate (—OMs, —OSO₂Me), and triflate (—OTf, —OSO₂(CF₃)).

Various leaving groups are discussed, for example, in ‘March's Advanced Organic Chemistry’, Wiley, 6^{th} Edition, pages 496-501 and 555-571 and in ‘Organic Chemistry’, Clayden et al, Oxford University Press, 1^{st} Edition, pages 407-445.

In some embodiments, X is halide.

In some embodiments, X is selected from —F, —Cl, —Br, and —I.

In some embodiments, in the electrophile addition step, the compound of formula (II) is treated with a base.

In some embodiments, in the electrophile addition step the compound of formula (II) is treated with a base prior to addition of the electrophile.

In some embodiments, the base is selected from sodium hydride, potassium hydride, calcium hydride, potassium carbonate, lithium diisopropyl amine, diisopropylethyl amine, and DBU (1,8-diazabicyclo(5.4.0)undec-7-ene).

In some embodiments, the base is sodium hydride.

In some embodiments, after reaction, the reaction mixture is concentrated.

In some embodiments, after reaction, the product is purified by chromatography.

In some embodiments, the alkylating agent is an aldehyde.

In some embodiments, the alkylating agent is formaldehyde.

In some cases, a compound with a less labile leaving group, such as a hydroxide or alkoxide, may be activated to form a better leaving group. For example this could be by protonation, by reaction with e.g. PBr₃, or via a reaction such as a Mitsunobu reaction, which uses a combination of a phosphine, for example triphenylphosphine (PPh₃), and an azodicarboxylate, for example DEAD or DIAD, to activate a hydroxide group to nucleophilic attack.

This is illustrated in the scheme below:

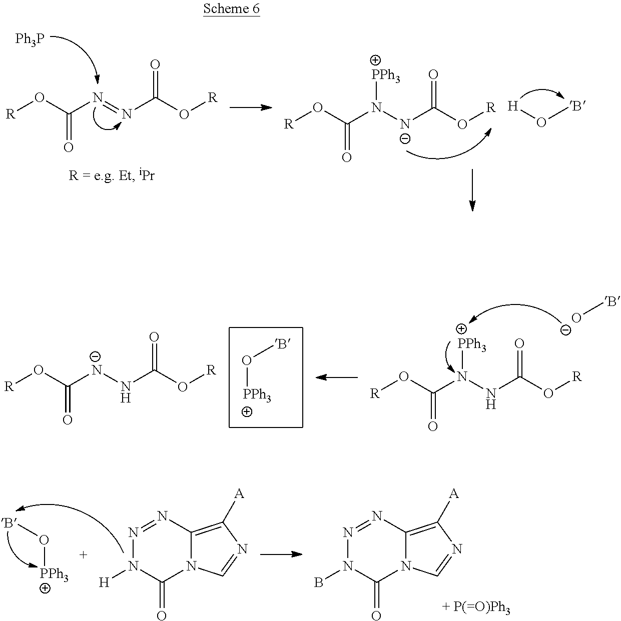

In some embodiments, the alkylation step comprises a Mitsunobu reaction.

In some embodiments, the alkylation step comprises a Mitsunobu reaction between a compound of formula (II) and an alcohol.

In some embodiments, the alkylation step comprises a Mitsunobu reaction between a compound of formula (II) and an alcohol of general formula B—OH.

In some embodiments, the alkylation step comprises a Mitsunobu reaction between a compound of formula (II) and an alcohol of general formula B—OH, in the presence of triphenylphosphine and an azodicarboxylate.

In some embodiments, the azodicarboxylate is selected from diethylazodicarboxylate (DEAD).

In some embodiments, the azodicarboxylate is selected from diisopropylazodicarboxylate (DIAD).

In some embodiments, the triphenylphosphine is polymer-supported.

In some embodiments, the alcohol is a C₁₋₆ alkyl alcohol and is optionally substituted.

In some embodiments, the alcohol is benzyl alcohol.

In some embodiments, the alcohol is glycidol.

After the alkylation step, the product may be further modified at the 3-position and/or the 8-position, to produce the final desired compound, which may be a compound of formula (I), as discussed further below.

### Modifications at the 3- and 8-Positions

After the alkylation step, the product may be further modified at the 3-position and/or the 8-position, to produce the final desired compound, which may be a compound of formula (I).

As will be appreciated by those skilled in the art, manipulation of the groups at the 3- and 8-positions may equally, or additionally, be made at other points in the synthesis, as required, for example to produce alternative and further ‘A’ substituents in the compounds of formulae (II), (III) and (V).

Further synthetic modifications to the group introduced at the 3-position may be made to create further analogues, using synthetic techniques and transformations which are known in the art and which can readily be devised by the skilled person.

For example, a 3-hydroxymethyl compound as shown below may be used to prepare a range of other 3-substituted compounds, by reaction with a suitable halide (e.g., R—X, where X is, for example, —I), for example, in the presence of a suitable base. A wide variety of halides is known and/or can be relatively easily prepared. An example of this method is illustrated in the following scheme.

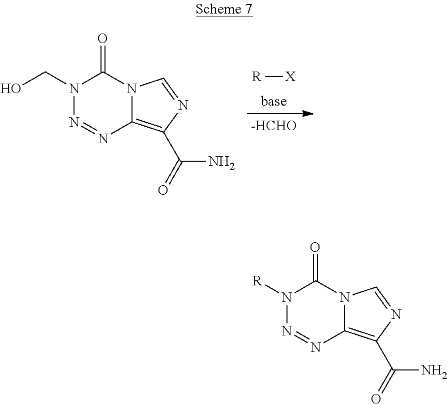

Examples of compounds which could be made using this method include, but are not limited to, compounds where R is selected from groups such as benzyl, p-methoxybenzyl, methyl, ethyl, propyl, propargyl, or methoxymethyl (MOM).

Modifications at the 8-position may be made, for example, by starting from the corresponding carboxamide. Suitable methods for reaction of a carboxamide group, to produce other functional groups, are known in the art. Methods for modification of a carboxamide group at the 8-position of certain temozolomide analogues are described in co-pending U.S. patent application 61/219,575. Derivatives that can be prepared from the 8-carboxamide include, but are not limited to, those discussed below.

For example, an 8-carboxy compound may be synthesised from the corresponding 8-carboxamide by treatment with, for example, trifluoroacetic acid and sodium nitrite in water.

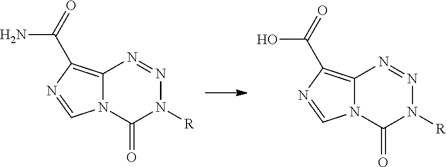

The carboxylic acid may be further derivatised, for example as discussed below.

A C-8 thioamide group may be prepared from a C-8 carboxamide by, for example, treatment with Belleau's reagent or with phosphorous pentasulphide.

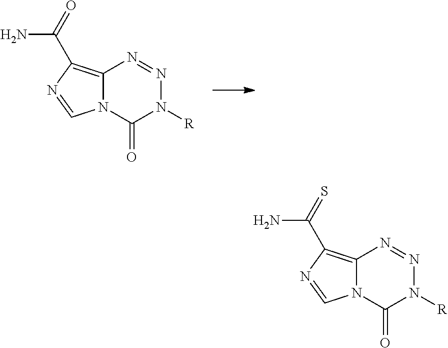

The thioamide may be further derivatised, for example as discussed below.

A C-8 imidamide group may be prepared from the corresponding C-8 thioamide, for example by treatment with methyl iodide followed by an appropriate amine.

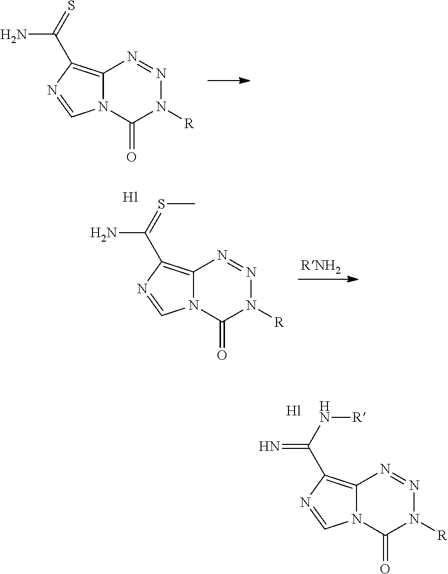

A C-8 thiazole group may be prepared from a C-8 thioamide, for example, by reaction with an appropriate α-bromo ketone.

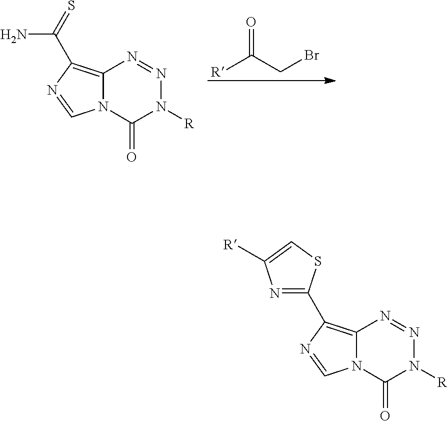

A C-8 oxazole group may be prepared from a C-8 carboxamide, for example, by conversion to the carboxylic acid as described above followed by reaction with an appropriate α-aminoketone hydrochloride.

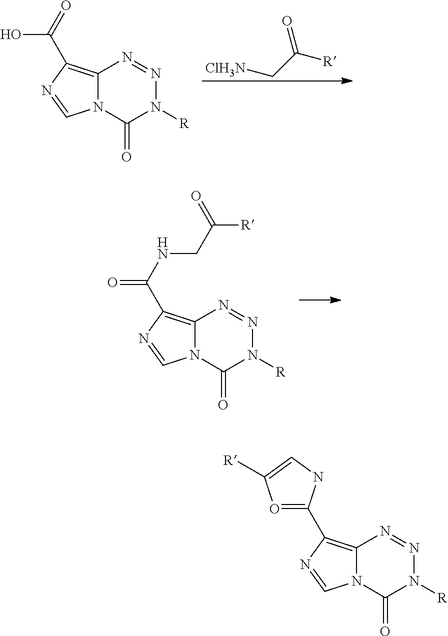

A C-8 oxadiazole group may be prepared from a C-8 carboxylic acid, for example, by reaction with an appropriate α-hydrazidoketone.

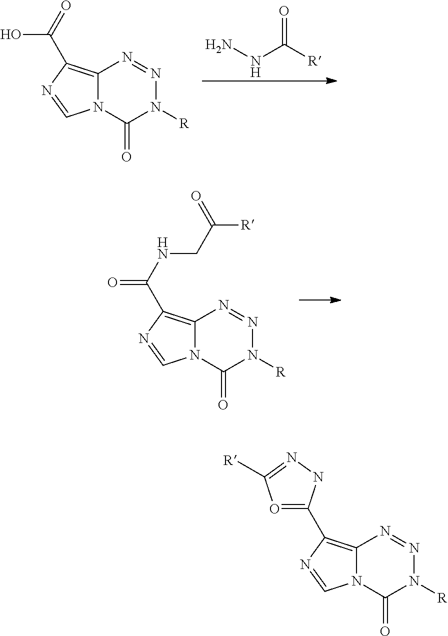

A C-8 imidazole group may be prepared from a C-8 carboxamide group, for example, by conversion to the corresponding C-8 thioamide followed by reaction with treatment with methyl iodide followed by an appropriate α-aminoketone hydrochloride.

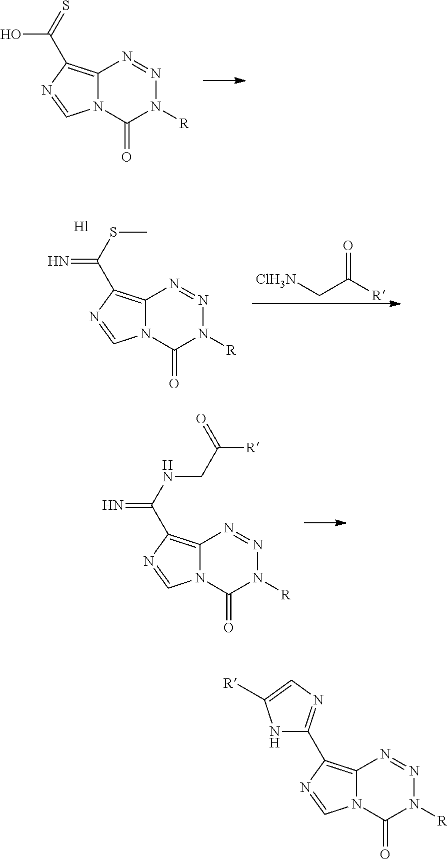

A C8 benzoxazole group may be prepared from the corresponding C-8 carboxylic acid, for example by treatment with triethylamine and 2-aminophenol, followed by an azodicarboxylate and triphenylphosphine.

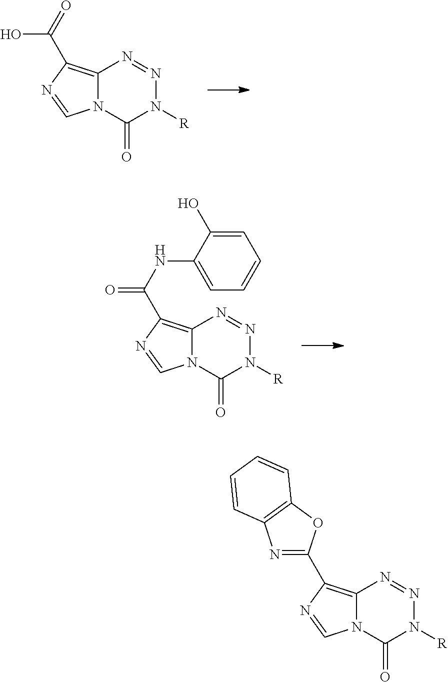

A C-8 benzimidazole group may be prepared from the corresponding C8-carboxylic acid, for example by coupling the acid with phenylene diamine and then cyclising.

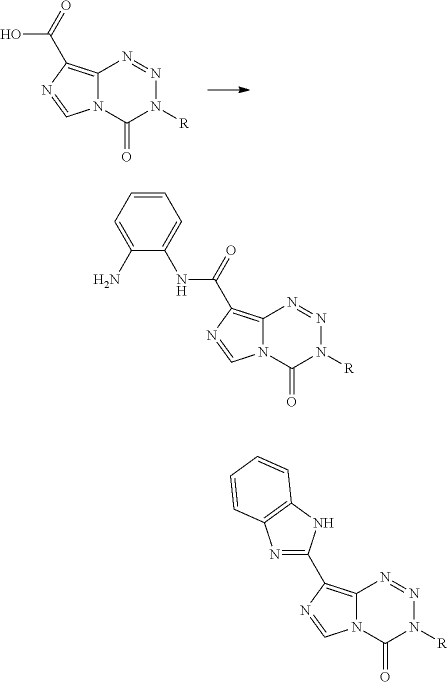

A C-8 aminooxadiazole may be prepared from the corresponding C8-carboxylic acid, for example by coupling with a thiosemicarbazide.

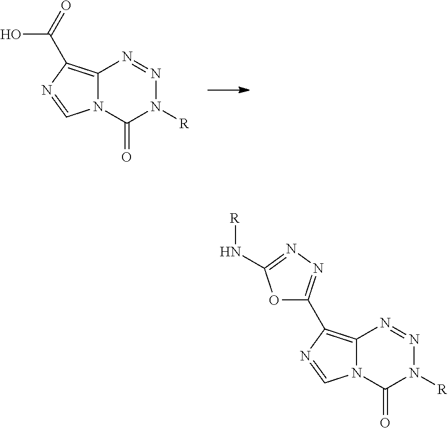

C8 substituted carboxamides may be prepared from the corresponding carboxamides by treatment with a base such as sodium hydride followed by an alkyl halide.

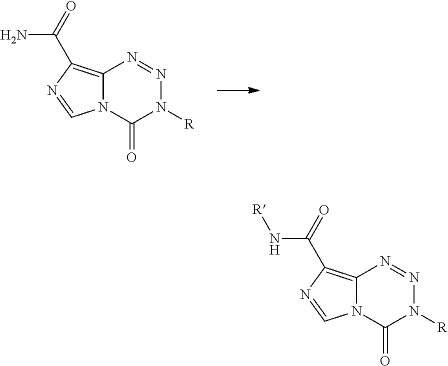

Alternatively, C8 substituted carboxamides may be prepared from the corresponding C8-carboxylic acids, for example by coupling with an appropriate amine.

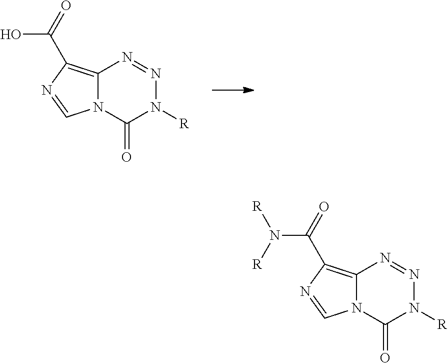

A C-8 benzothiazole group may be prepared from the corresponding N-phenyl C-8 carboxamide for example by conversion to the corresponding thioamide and finally cyclisation.

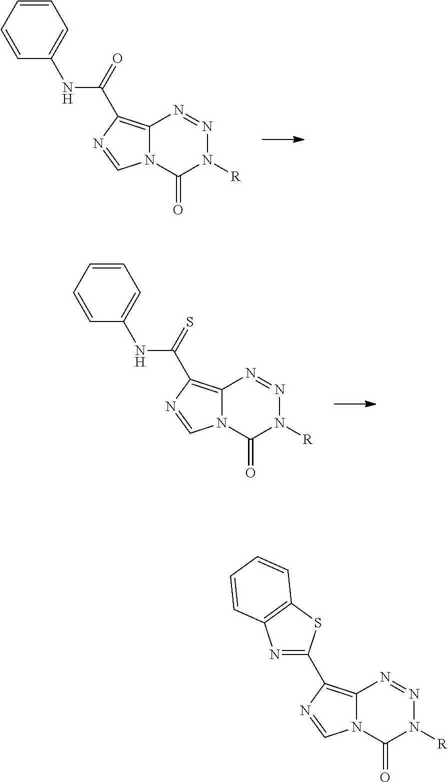

### Further Definitions and Embodiments

In the compounds, methods, uses, and processes of the invention, the compounds of formulae (I), (II), (III), (IV) and (V) are as defined herein.

### Compounds (I), (II), (III)

In some embodiments the compound of formula (I) is a compound of formula:

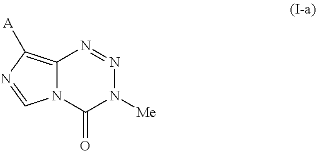

In some embodiments the compound of formula (I) is a compound of formula:

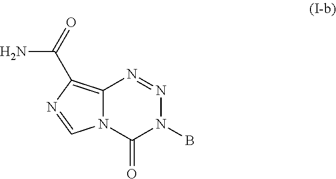

In some embodiments, the compound of formula (II) is a compound of formula:

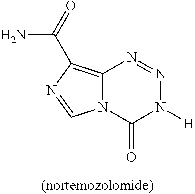 [SMILES: NC(=O)c1ncn2c(=O)[nH]nnc12]

In some embodiments, the compound of formula (III) is a compound of formula:

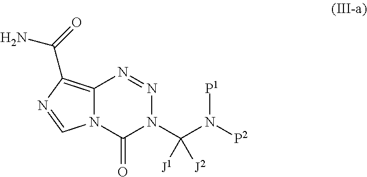

In some embodiments, the compound of formula (III) is a compound of formula:

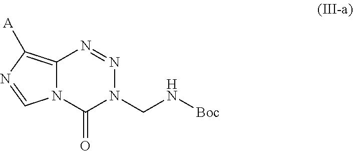

### Groups A, B, J¹, J², P¹ and P²

In the compounds of formulae (I), (II), (III), (IV) and (V) the groups A, B, J¹, J², P¹ and P² are as defined herein.

The groups A, B, -A¹, -A², -A³, -A⁴, -A⁵, -A⁶, -A⁷, -B¹, -B², -B³, -B⁴, -B⁵, -B⁶, -B⁷, -B⁸, -B⁹, -B¹⁰, -B¹¹, B¹², -B¹³, -B¹⁴, J¹, J², P¹ and a P² are defined herein as independent variables. As will be recognised by those skilled in the art, any compatible combination of these groups and substituents may be utilised in the methods and compounds of the present invention.

All compatible combinations of these and other defined variables are specifically embraced by the present invention, and are disclosed herein as if each and every combination were individually and explicitly recited.

### The Group A

A is independently selected from -A¹, -A², -A³, -A⁴, -A⁵, -A⁶, and -A⁷ wherein:
-A¹ is independently C₅₋₁₂heteroaryl, and is optionally substituted;
-A² is independently thioamido or substituted thioamido;
-A³ is independently imidamido, substituted imidamido,
-A⁴ is independently hydroxamic acid or hydroxamate;
-A⁵ is independently carboxamide or substituted carboxamide;
-A⁶ is independently aliphatic C₂₋₆alkenyl and is optionally substituted; and
-A⁷ is independently carboxy or C₁₋₄alkyl-carboxylate.

### A—Groups -A¹, -A², -A³, -A⁴, -A⁵, -A⁶, and -A⁷

A is independently -A¹, -A², -A³, -A⁴, -A⁵, -A⁶, or -A⁷.

In some embodiments, the group A is a group which is obtainable by derivatisation of a carboxamide (—C(═O)NH₂) group. This derivatisation may be done at any convenient stage of the synthesis.

### A¹—8-Heteroaryl Groups

In some embodiments, A is -A¹, where -A¹ is independently C₅₋₁₂heteroaryl, and is optionally substituted.

In some embodiments -A¹ is independently furanyl, thienyl, pyrrolyl, imidazolyl, pyrazolyl, triazolyl, tetrazolyl, oxazolyl, isoxazolyl, thiazolyl, isothiazolyl, pyridyl, pyrimidinyl, pyridazinyl, indolyl, isoindolyl, benzofuranyl, isobenzofuranyl, benzothienyl, isobenzothienyl, indazolyl, benzimidazolyl, benzothiazolyl, benzoisothiazolyl, benzoxazolyl, benzoisoxazolyl, quinolinyl, isoquinolinyl, cinnolinyl, or quinazolinyl, and is optionally substituted.

In some embodiments, -A¹ is independently C₅₋₆heteroaryl, and is optionally substituted.

In some embodiments, -A¹ is independently furanyl, thienyl, pyrrolyl, imidazolyl, pyrazolyl, triazolyl, tetrazolyl, oxazolyl, isoxazolyl, thiazolyl, isothiazolyl, pyridyl, pyrimidinyl, or pyridazinyl, and is optionally substituted.

In some embodiments, -A¹ is independently C₉₋₁₀heteroaryl, and is optionally substituted.

In some embodiments, -A¹ is independently indolyl, isoindolyl, benzofuranyl, isobenzofuranyl, benzothienyl, isobenzothienyl, indazolyl, benzimidazolyl, benzothiazolyl, benzoisothiazolyl, benzoxazolyl, benzoisoxazolyl, quinolinyl, isoquinolinyl, cinnolinyl, or quinazolinyl, and is optionally substituted.

In some embodiments, -A¹ is independently benzimidazolyl, benzothiazolyl, or benzoxazolyl, and is optionally substituted.

In some embodiments, -A¹ is unsubstituted.

In some embodiments, -A¹ is substituted with one or more groups independently selected from:
—F, —Cl, —Br, —I,
—R^{Z1}, —CF₃,
—OH, —OR^{Z1}, —OCF₃,
—SR^{X1},

—NH₂, —NHR^{Z1}, —NR^{Z1}₂, pyrrolidino, piperidino, morpholino, piperizino, (N—C₁₋₄alkyl)-piperizino,
—C(═O)OH, —C(═O)OR^{Z1},
—C(═O)R^{Z1},
—OC(═O)R^{Z1},
—C(═O)NH₂, —C(═O)NHR²¹, —C(═O)NR^{Z1}₂, —C(═O)-pyrrolidino, —C(═O)-piperidino, —C(═O)-morpholino, —C(═O)-piperizino, —C(═O)—(N—C₁₋₄alkyl)-piperizino,
—NHC(═O)R^{Z1}, —NR^{Z1}C(═O)R^{Z1}, —OC(═O)NH₂, —OC(═O)NHR^{Z1}, —OC(═O)NR^{Z1}₂, —OC(═O)-pyrrolidino, —OC(═O)-piperidino, —OC(═O)-morpholino, —OC(═O)-piperizino, (N—C₁₋₄alkyl)-piperizino,
—NHC(═O)OH, —NHC(═O)OR^{Z1}, —NR^{Z1}C(═O)OR^{Z1},
—NHC(═O)NH₂, —NHC(═O)NHR^{Z1}, —NHC(═O)NR^{Z1}₂, —NHC(═O)-pyrrolidino, —NHC(═O)-piperidino, —NHC(═O)-morpholino, —NHC(═O)-piperizino, —NHC(═O)—(N—C₁₋₄alkyl)-piperizino,
—NO₂, and —CN,
wherein each —R^{Z1} is independently saturated aliphatic C₁₋₄alkyl, aliphatic C₃₋₆alkynyl, saturated C₃₋₆cycloalkyl, C₅₋₆heteroaryl, -Ph, or —CH₂-Ph,
wherein each of said C₃₋₆cycloalkyl, C₅₋₆heteroaryl, and -Ph is optionally substituted, for example, with one or more substituents selected from —F, —Cl, —Br, —I, —R^{Z1A}, —CF₃, —OH, —OR^{Z1A}, and —OCF₃,
wherein each —R^{Z1A} is independently saturated aliphatic C₁₋₄alkyl,
and additionally wherein two adjacent substituents may together form —O—CH₂—O— or —O—CH₂CH₂—O—.

### A²—8-Thioamide Groups

In some embodiments, -A is -A², wherein -A² is independently thioamido or substituted thioamido.

In some embodiments, -A² is independently:
—C(═S)NH₂, —C(═S)NHR^{Z2}, —C(═S)NR^{Z2}₂, —C(═S)-pyrrolidino, —C(═S)-piperidino, —C(═S)-morpholino, —C(═S)-piperizino, or —C(═S)—(N—C₁₋₄alkyl)-piperizino,
wherein:
—R^{Z2} is independently saturated aliphatic C₁₋₄alkyl, saturated C₃₋₆cycloalkyl, C₅₋₆heteroaryl, —CH₂—C₅₋₆heteroaryl, -Ph, or —CH₂-Ph,
wherein each of said C₃₋₆cycloalkyl, C₅₋₆heteroaryl, and -Ph is optionally substituted.

In some embodiments, each of said C₃₋₆cycloalkyl, C₅₋₆heteroaryl, and -Ph is optionally substituted with one or more groups independently selected from:
—F, —Cl, —Br, —I,
—R^{Z2A}, —CF₃,
—OH, —OR^{Z2A},
—SR^{Z2A},
—NH₂, —NHR^{Z2A}, —NR^{Z2A}₂, pyrrolidino, piperidino, morpholino, piperizino, (N—C₁₋₄alkyl)-piperizino,
—C(═O)OH, —C(═O)OR^{Z2A},
—C(═O)R^{Z2A},
—OC(═O)R^{Z2A},
—C(═O)NH₂, —C(═O)NHR^{Z2A}, —C(═O)NR^{Z2A}₂, —C(═O)-pyrrolidino, —C(═O)-piperidino, —C(═O)-morpholino, —C(═O)-piperizino, —C(═O)—(N—C₁₋₄alkyl)-piperizino,
—NHC(═O)R^{Z2A}, —NR^{Z2A}C(═O)R^{Z2A},
—OC(═O)NH₂, —OC(═O)NHR^{Z2A}, —OC(═O)NR^{Z2A}₂, —OC(═O)-pyrrolidino, —OC(═O)-piperidino, —OC(═O)-morpholino, —OC(═O)-piperizino, (N—C₁₋₄alkyl)-piperizino,
—NHC(═O)OH, —NHC(═O)OR^{Z2A}, —NR^{Z2A}C(═O)OR^{Z2A},
—NHC(═O)NH₂, —NHC(═O)NHR^{Z2A}, —NHC(═O)NR^{Z2A}₂, —NHC(═O)-pyrrolidino, —NHC(═O)-piperidino, —NHC(═O)-morpholino, —NHC(═O)-piperizino, —NHC(═O)—(N—C₁₋₄alkyl)-piperizino,
—NO₂, and —CN,
wherein each —R^{Z2A} is independently saturated aliphatic C₁₋₄alkyl, saturated C₃₋₆cycloalkyl, -Ph, or —CH₂-Ph,
wherein each C₃₋₆cycloalkyl and -Ph is optionally substituted with one or more substituents selected from —F, —Cl, —Br, —I, —R^{Z2B}, —CF₃, —OH, —OR^{Z2B}, and —OCF₃,
wherein each —R^{Z2B} is independently saturated aliphatic C₁₋₄alkyl.

In some embodiments -A² is independently —C(═S)NH₂, —C(═S)NHR^{Z2}, or —C(═S)NR^{Z2}₂.

In some embodiments -A² is independently —C(═S)NH₂ or —C(═S)NHR^{Z2}.

In some embodiments -A² is independently —C(═S)NH₂.

In some embodiments —R^{Z2}, if present, is independently saturated aliphatic C₁₋₄alkyl, saturated C₃₋₆cycloalkyl, C₅₋₆heteroaryl, —CH₂—C₅₋₈heteroaryl, -Ph, or —CH₂-Ph, wherein each of said C₃₋₆cycloalkyl, C₅₋₆heteroaryl, and -Ph is optionally substituted with one or more groups selected from: —F, —Cl, —Br, —I, —R^{Z2A}, —CF₃, —OH, —OR^{Z2A}, and —OCF₃

### A³—8-Imidamide Groups

In some embodiments, -A is independently -A³, wherein -A³ is independently imidamido or substituted imidamido.

In some embodiments, -A³ is independently:
—C(═NH)NH₂, —C(═NH)NHR^{Z3}, or —C(═NH)NR^{Z3}₂, —C(═NH)-pyrrolidino, —C(═NH)-piperidino, —C(═NH)-morpholino, —C(═NH)-piperizino, —C(═NH)—N—C₁₋₄alkyl)-piperizino, —C(═N—R^{Z3})NH₂, —C(═N—R^{Z3})NHR^{Z3}, —C(═N—R^{Z3})NR^{Z3}₂, —C(═N—R^{Z3})-pyrrolidino, —C(═N—R^{Z3})-piperidino, —C(═N—R^{Z3})-morpholino, —C(═N—R^{Z3})-piperizino, —C(═N—R^{Z3})—N—C₁₋₄alkyl)-piperizino;
wherein:
—R²³ is independently saturated aliphatic C₁₋₄alkyl, saturated C₃₋₆cycloalkyl, C₅₋₆heteroaryl, —CH₂—C₅₋₆heteroaryl, -Ph, or —CH₂-Ph,
wherein each of said C₁₋₄alkyl, C₃₋₆cycloalkyl, C₅₋₆heteroaryl, and -Ph is optionally substituted.

In some embodiments, -A³ is independently:
—C(═NH)NH₂, —C(═NH)NHR^{Z3}, or —C(═NH)NR^{Z3}₂, —C(═NH)-pyrrolidino, —C(═NH)-piperidino, —C(═NH)-morpholino, —C(═NH)-piperizino, —C(═NH)—N—C₁₋₄alkyl)-piperizino; —C(═N—R^{Z3})NH₂, —C(═N—R^{Z3})NHR^{Z3}, —C(═N—R^{Z3})NR^{Z3}₂, —C(═N—R^{Z3})-pyrrolidino, —C(═N—R^{Z3})-piperidino, —C(═N—R^{Z3})-morpholino, —C(═N—R^{Z3})-piperizino, or —C(═N—R^{Z3})—N—C₁₋₄alkyl)-piperizino, wherein:
—R^{Z3} is independently saturated aliphatic C₁₋₄alkyl, saturated C₃₋₆cycloalkyl, C₅₋₆heteroaryl, —CH₂—C₅₋₆heteroaryl, -Ph, or —CH₂-Ph,
wherein each of said C₁₋₄alkyl, C₃₋₆cycloalkyl, C₅₋₆heteroaryl, and -Ph is optionally substituted.

In some embodiments, -A³ is independently:
—C(═N—R^{Z3})NH₂, —C(═N—R^{Z3})NHR^{Z3}, —C(═N—R^{Z3})NR^{Z3}₂, —C(═N—R^{Z3})-pyrrolidino, —C(═N—R^{Z3})-piperidino, —C(═N—R^{Z3})-morpholino, —C(═N—R^{Z3})-piperizino, or —C(═N—R^{Z3})—N—C₁₋₄alkyl)-piperizino, wherein:
—R^{Z3} is independently saturated aliphatic C₁₋₄alkyl, saturated C₃₋₆cycloalkyl, C₅₋₆heteroaryl, —CH₂—C₅₋₆heteroaryl, -Ph, or —CH₂-Ph,
wherein each of said C₁₋₄alkyl, C₃₋₆cycloalkyl, C₅₋₆heteroaryl, and -Ph is optionally substituted.

In some embodiments each of said C₁₋₄alkyl, C₃₋₆cycloalkyl, C₅₋₆heteroaryl, and -Ph is optionally substituted with one or more groups independently selected from:
—F, —Cl, —Br, —I,
—R^{Z3A}, —CF₃,
—OH, —OR^{Z3A}, —OCF₃,
—SR^{Z3A},
—NH₂, —NHR^{Z3A}, —NR^{Z3A}₂, pyrrolidino, piperidino, morpholino, piperizino, (N—C₁₋₄alkyl)-piperizino,
—C(═O)OH, —C(═O)OR^{Z3A},
—C(═O)R^{Z3A},
—OC(═O)R^{Z3A},
—C(═O)NH₂, —C(═O)NHR^{Z3A}, —C(═O)NR^{Z3A}₂, —C(═O)-pyrrolidino, —C(═O)-piperidino, —C(═O)-morpholino, —C(═O)-piperizino, —C(═O)—(N—C₁₋₄alkyl)-piperizino,
—NHC(═O)R^{Z3A}, —NR^{Z3A}C(═O)R^{Z3A},
—OC(═O)NH₂, —OC(═O)NHR^{Z3A}, —OC(═O)NR^{Z3A}₂, —OC(═O)-pyrrolidino, —OC(═O)-piperidino, —OC(═O)-morpholino, —OC(═O)-piperizino, (N—C₁₋₄alkyl)-piperizino,
—NHC(═O)OH, —NHC(═O)OR^{Z3A}, —NR^{Z3A}C(═O)OR^{Z3A},
—NHC(═O)NH₂, —NHC(═O)NHR^{Z3A}, —NHC(═O)NR^{Z3A}₂, —NHC(═O)-pyrrolidino, —NHC(═O)-piperidino, —NHC(═O)-morpholino, —NHC(═O)-piperizino, —NHC(═O)—(N—C₁₋₄alkyl)-piperizino,
—NO₂, and —CN,
wherein each —R^{Z3A} is independently saturated aliphatic C₁₋₄alkyl, saturated C₃₋₆cycloalkyl, -Ph, or —CH₂-Ph, wherein each of said C₃₋₆cycloalkyl and -Ph is optionally substituted with one or more substituents selected from —F, —Cl, —Br, —I, —R^{Z3B}, —CF₃, —OH, —OR^{Z3B}, and —OCF₃, wherein each —R^{Z3B} is independently saturated aliphatic C₁₋₄alkyl.

In some embodiments -A³ is independently —C(═NH)NH₂, —C(═NH)NHR^{Z3}, or —C(═NH)NR^{Z3}₂.

In some embodiments -A³ is independently —C(═NH)NH₂ or —C(═NH)NHR^{Z3}.

In some embodiments -A³ is independently —C(═NH)NHR^{Z3}.

In some embodiments, —R^{Z3}, where present, is independently saturated aliphatic C₁₋₄alkyl or saturated C₃₋₆cycloalkyl.

In some embodiments, —R^{Z3}, where present, is independently selected from -Me, -Et, -nPr, -iPr, -nBu, -sBu, -iBu, and -tBu.

### A⁴—8-Hydroxamate Groups

In some embodiments, -A is independently -A⁴, wherein -A⁴ is independently hydroxamic acid or hydroxamate.

In some embodiments -A⁴ is independently:
—C(═O)—NH—OH, —C(═O)—NR^{Z4}—OH, —C(═O)—NH—OR^{Z4}, —C(═O)—NR^{Z4}—OR^{Z4},
wherein:
—R^{Z4} is independently saturated aliphatic C₁₋₄alkyl, saturated C₃₋₆cycloalkyl, C₅₋₆heteroaryl, —CH₂—C₅₋₆heteroaryl, -Ph, or —CH₂-Ph,
wherein each of said C₃₋₆cycloalkyl, C₅₋₆heteroaryl, and -Ph is optionally substituted.

In some embodiments, each of said C₃₋₆cycloalkyl, C₅₋₆heteroaryl, and -Ph is optionally substituted with one or more groups independently selected from:
—F, —Cl, —Br, —I,
—R^{Z4A}, —CF₃,
—OH, —OR^{Z4A}, —OCF₃,
—SR^{Z4A},
—NH₂, —NHR^{Z4A}, —NR^{Z4A}₂, pyrrolidino, piperidino, morpholino, piperizino, (N—C₁₋₄alkyl)-piperizino,
—C(═O)OH, —C(═O)OR^{Z4A},
—C(═O)R^{Z4A},
—OC(═O)R^{Z4A},
—C(═O)NH₂, —C(═O)NHR^{Z4A}, —C(═O)NR^{Z4A}₂, —C(═O)-pyrrolidino, —C(═O)-piperidino, —C(═O)-morpholino, —C(═O)-piperizino, —C(═O)—(N—C₁₋₄alkyl)-piperizino,
—NHC(═O)R^{Z4A}, —NR^{Z4A}C(═O)R^{Z4A},
—OC(═O)NH₂, —OC(═O)NHR^{Z4A}, —OC(═O)NR^{Z4A}₂, —OC(═O)-pyrrolidino, —OC(═O)-piperidino, —OC(═O)-morpholino, —OC(═O)-piperizino, (N—C₁₋₄alkyl)-piperizino,
—NHC(═O)OH, —NHC(═O)OR^{Z4A}, —NR^{Z4A}C(═O)OR^{Z4A},
—NHC(═O)NH₂, —NHC(═O)NHR^{Z4A}, —NHC(═O)NR^{Z4A}₂, —NHC(═O)-pyrrolidino, —NHC(═O)-piperidino, —NHC(═O)-morpholino, —NHC(═O)-piperizino, —NHC(═O)—(N—C₁₋₄alkyl)-piperizino,
—NO₂, and —CN,
wherein each —R^{Z4A} is independently saturated aliphatic C₁₋₄alkyl, saturated C₃₋₆cycloalkyl, -Ph, or —CH₂-Ph,
wherein each of said C₃₋₆cycloalkyl and -Ph is optionally substituted with one or more substituents selected from —F, —Cl, —Br, —I, —R^{Z4B}, —CF₃, —OH, —OR^{Z4B}, and —OCF₃,
wherein each —R^{Z4B} is independently saturated aliphatic C₁₋₄alkyl.

In some embodiments -A⁴ is independently —C(═O)—NH—OH or —C(═O)—NH—OR^{Z4}.

In some embodiments -A⁴ is independently —C(═O)—NH—OR^{Z4}.

In some embodiments, —R^{Z4}, if present, is independently saturated aliphatic C₁₋₄alkyl, saturated C₃₋₆cycloalkyl, -Ph, or —CH₂-Ph.

In some embodiments, —R^{Z4}, if present, is independently saturated aliphatic C₁₋₄alkyl, -Ph, or —CH₂-Ph.

In some embodiments, -A⁴ is independently —C(═O)—NH—OH.

In some embodiments, -A⁴ is independently —C(═O)—NH—O—CH₂-Ph.

### A⁵—8-Carboxamide and 8-Substituted Carboxamide Groups

In some embodiments, -A is independently -A⁵, wherein -A⁵ is independently carboxamide or substituted carboxamide.

In some embodiments -A⁵ is independently:
—C(═O)—NH₂, —C(═O)—NHR^{Z5}, —C(═O)—NR^{Z5}₂, —C(═O)-pyrrolidino, —C(═O)-piperidino, —C(═O)-morpholino, —C(═O)-piperizino, or —C(═O)—(N—C₁₋₄alkyl)-piperizino,
wherein:
—R^{Z5} is independently saturated aliphatic C₁₋₄alkyl, saturated C₃₋₆cycloalkyl, C₅₋₆heteroaryl, —CH₂—C₅₋₆heteroaryl, -Ph, or —CH₂-Ph,
wherein each of said C₃₋₆cycloalkyl, C₅₋₆heteroaryl, and -Ph is optionally substituted.

In some embodiments, each of said C₃₋₆cycloalkyl, C₅₋₆heteroaryl, and -Ph is optionally substituted with one or more groups independently selected from:
—F, —Cl, —Br, —I,
—R^{Z5A}, —CF₃,
—OH, —OR^{Z5A}, —OCF₃,
—SR^{Z5A},
—NH₂, —NHR^{Z5A}, —NR^{Z5A}₂, pyrrolidino, piperidino, morpholino, piperizino, (N—C₁₋₄alkyl)-piperizino,
—C(═O)OH, —C(═O)OR^{Z5A},
—C(═O)R^{Z5A},
—OC(═O)R^{Z5A},
—C(═O)NH₂, —C(═O)NHR^{Z5A}, —C(═O)NR^{Z5A}₂, —C(═O)-pyrrolidino, —C(═O)-piperidino, —C(═O)-morpholino, —C(═O)-piperizino, —C(═O)—(N—C₁₋₄alkyl)-piperizino,
—NHC(═O)R^{Z5A}, —NR^{Z5A}C(═O)R^{Z5A},
—OC(═O)NH₂, —OC(═O)NHR^{Z5A}, —OC(═O)NR^{Z5A}₂, —OC(═O)-pyrrolidino, —OC(═O)-piperidino, —OC(═O)-morpholino, —OC(═O)-piperizino, (N—C₁₋₄alkyl)-piperizino,
—NHC(═O)OH, —NHC(═O)OR^{Z5A}, —NR^{Z5A}C(═O)OR^{Z5A},
—NHC(═O)NH₂, —NHC(═O)NHR^{Z5A}, —NHC(═O)NR^{Z5A}₂, —NHC(═O)-pyrrolidino, —NHC(═O)-piperidino, —NHC(═O)-morpholino, —NHC(═O)-piperizino, —NHC(═O)—(N—C₁₋₄alkyl)-piperizino,
—NO₂, and —CN,
wherein each —R^{Z5A} is independently saturated aliphatic C₁₋₄alkyl, saturated C₃₋₆cycloalkyl, -Ph, or —CH₂-Ph,
wherein each of said C₃₋₆cycloalkyl and -Ph is optionally substituted with one or more substituents selected from —F, —Cl, —Br, —I, —R^{Z5B}, —CF₃, —OH, —OR^{Z5B}, and —OCF₃, and
wherein each —R^{Z5B} is independently saturated aliphatic C₁₋₄alkyl.

In some embodiments -A⁵ is independently —C(═O)NH₂, —C(═O)—NHR^{Z5} or —C(═O)—NR^{Z5}₂.

In some embodiments -A⁵ is independently —C(═O)NH₂ or —C(═O)—NHR^{Z5}.

In some embodiments —R^{Z5}, if present, is independently -Ph or —CH₂-Ph.

In some embodiments —R^{Z5}, if present, is independently saturated aliphatic C₁₋₄alkyl.

In some embodiments -A⁵ is independently —C(═O)—NHPh.

In some embodiments -A⁵ is independently —C(═O)NH₂.

### A⁶—C-8 Alkene Groups

In some embodiments, -A is independently -A⁶, wherein -A⁶ is independently aliphatic C₂₋₆alkenyl, and is optionally substituted.

In some embodiments, -A⁶ is independently -L⁶-R^{Z6},
wherein:
-L⁶- is independently aliphatic C₂₋₆alkenyl, and
—R^{Z6} is independently C₅₋₆heteroaryl or -Ph,
wherein each of said C₅₋₆heteroaryl and -Ph is optionally substituted.

In some embodiments, each of said C₅₋₆heteroaryl and -Ph is optionally substituted with one or more groups independently selected from:
—F, —Cl, —Br, —I,
—R^{Z6A}, —CF₃,
—OH, —OR^{Z6A}, —OCF₃,
—SR^{Z6A},
—NH₂, —NHR^{Z6A}, —NR^{Z6A}₂, pyrrolidino, piperidino, morpholino, piperizino, (N—C₁₋₄alkyl)-piperizino,
—C(═O)OH, —C(═O)OR^{Z6A},
—C(═O)R^{Z6A},
—OC(═O)R^{Z6A},
—C(═O)NH₂, —C(═O)NHR^{Z6A}, —C(═O)NR^{Z6A}₂, —C(═O)-pyrrolidino, —C(═O)-piperidino, —C(═O)-morpholino, —C(═O)-piperizino, —C(═O)—(N—C₁₋₄alkyl)-piperizino,
—NHC(═O)R^{Z6A}, —NR^{Z6A}X(═O)R^{Z6A},
—OC(═O)NH₂, —OC(═O)NHR^{Z6A}, —OC(═O)NR^{Z6A}₂, —OC(═O)-pyrrolidino, —OC(═O)-piperidino, —OC(═O)-morpholino, —OC(═O)-piperizino, (N—C₁₋₄alkyl)-piperizino,
—NHC(═O)OH, —NHC(═O)OR^{Z6A}, —NR^{Z6A}C(═O)OR^{Z6A},
—NHC(═O)NH₂, —NHC(═O)NHR^{Z6A}, —NHC(═O)NR^{Z6A}₂, —NHC(═O)-pyrrolidino, —NHC(═O)-piperidino, —NHC(═O)-morpholino, —NHC(═O)-piperizino, —NHC(═O)—(N—C₁₋₄alkyl)-piperizino,
—NO₂, and —CN,
wherein each —R^{Z6A} is independently saturated aliphatic C₁₋₄alkyl, saturated C₃₋₆cycloalkyl, -Ph, or —CH₂-Ph,
wherein each C₃₋₆cycloalkyl and -Ph is optionally substituted with one or more substituents selected from —F, —Cl, —Br, —I, —R^{Z6A}, —CF₃, —OH, —OR^{Z6B}, and —OCF₃,
wherein each —R^{Z6B} is independently saturated aliphatic C₁₋₄alkyl.

### A⁷—8-Carboxy Groups

In some embodiments, A is -A⁷, wherein -A⁷ is independently carboxy or C₁₋₄alkyl-carboxylate.

In some embodiments, -A⁸ is independently selected from:
—CO₂H or CO₂R^{Z8A}
wherein each —R^{Z8A} is independently saturated aliphatic C₁₋₆alkyl, saturated C₃₋₆cycloalkyl, -Ph, or —CH₂-Ph, wherein each C₃₋₆cycloalkyl and -Ph is optionally substituted.

In some embodiments, each said C₃₋₆cycloalkyl and -Ph is optionally substituted with one or more substituents selected from —F, —Cl, —Br, —I, —R^{Z8B}, —CF₃, —OH, —OR^{Z8B}, and —OCF₃, wherein each —R^{Z6B} is independently saturated aliphatic C₁₋₄alkyl.

In some embodiments, -A⁸ is independently —CO₂H.

In some embodiments, -A⁸ is independently CO₂R^{Z8A} wherein each —R^{Z8A} is independently saturated aliphatic C₁₋₆alkyl, saturated C₃₋₆cycloalkyl, -Ph, or —CH₂-Ph, and each C₃₋₆cycloalkyl and -Ph is optionally substituted.

In some embodiments, R^{Z8A}, where present, is independently saturated aliphatic C₁₋₆alkyl.

In some embodiments, R^{Z8A}, where present, is independently saturated aliphatic C₁₋₄alkyl.

In some embodiments, R^{Z8A}, where present, is independently selected from -Me, -Et, -nPr, -iPr, -nBu, -iBu, and -tBu.

In some embodiments, R^{Z8A}, where present, is independently selected from -Me and -Et.

### The Group B

B is independently selected from -B¹, -B², -B³, -B⁴, -B⁵, -B⁶, -B⁷, -B⁸, -B⁹, -B¹⁰, -B¹¹, B¹², -B¹³, -B¹⁴, -B¹⁵ and -B¹⁶

wherein:
-B¹ is independently saturated aliphatic C₁₋₆alkyl;
-B² is independently aliphatic C₂₋₆alkynyl;
-B³ is independently mercapto-C₁₋₄alkyl, sulfanyl-C₁₋₄alkyl, sulfinyl-C₁₋₄alkyl, or sulfonyl-C₁₋₄alkyl, or sulfonyloxy-C₁₋₄alkyl;
-B⁴ is independently hydroxy-C₁₋₄alkyl or ether-C₁₋₄alkyl;
-B⁵ is independently phenyl-C₁₋₆alkyl or C₅₋₁₂heteroaryl-C₁₋₆alkyl, and is optionally substituted;
-B⁶ is independently acyl-C₁₋₆alkyl, carboxy-C₁₋₆alkyl, oxyacyl-C₁₋₆alkyl, or acyloxy-C₁₋₆alkyl;
-B⁷ is independently amido-C₁₋₄alkyl or substituted amido-C₁₋₄alkyl;
-B⁸ is independently C₃₋₆cycloalkyl, C₃₋₆cycloalkyl-C₁₋₄alkyl, C₃₋₆heterocyclyl, or C₃₋₆heterocyclyl-C₁₋₄alkyl, and is optionally substituted;
-B⁹ is independently halo-C₁₋₆alkyl;
-B¹⁰ is independently nitro-C₁₋₆alkyl;
-B¹¹ is independently cyano-C₁₋₆alkyl;
-B¹² is independently phosphate-C₁₋₆alkyl;
-B¹³ is independently carbamate-C₁₋₆alkyl; and
-B¹⁴ is independently oxime-C₁₋₆alkyl.
   B—Groups -B¹, -B², -B³, -B⁴, -B⁵, -B⁶, -B⁷, -B⁸, -B⁹, -B¹⁰, -B¹¹, B¹², -B¹³, and -B¹⁴

### B¹—3-Alkyl Groups

In some embodiments, B^{A} is B¹, wherein B¹ is independently saturated aliphatic C₁₋₆alkyl.

In some embodiments, B is B¹, wherein B¹ is independently saturated aliphatic C₁₋₄alkyl.

In some embodiments, -B¹ is independently -Me, -Et, -nPr, -iPr, -nBu, -iBu, or -tBu.

In some embodiments, -B¹ is independently -Et or Me.

In some embodiments, -B¹ is independently -Me.

### B²—3-Alkynyl Groups

In some embodiments, B^{A} is B², wherein B² is independently C₂₋₆alkynyl.

As used herein, the term “alkynyl” relates to an aliphatic hydrocarbyl group (i.e., a group having only carbon atoms and hydrogen atoms) having at least one carbon-carbon triple bond.

In some embodiments, B² is independently aliphatic C₂₋₆alkynyl.

In some embodiments, -B² is independently aliphatic C₃₋₅alkynyl.

In some embodiments, -B² is independently:
—C≡CH,
—C≡C—CH₃, —CH₂—C≡CH,
—C≡C—CH₂—CH₃, —C≡C—CH═CH₂, —C≡C—C≡CH,
—CH₂—CH₂—C≡CH, —CH═CH—C≡CH, —C≡C—C≡CH,
—CH₂—C≡C—CH₃, or
—CH(CH₃)—C≡CH.

In some embodiments, -B² is independently propargyl (—CH₂—C≡CH).

### B³—3-Sulfur-Alkyl Groups

In some embodiments, B^{A} is B³, wherein -B³ is independently mercapto-C₁₋₄alkyl, sulfonyl-C₁₋₄alkyl, sulfinyl-C₁₋₄alkyl, or sulfonyl-C₁₋₄alkyl.

In some embodiments, -B³ is independently:
-L^{Y3}-SH, -L^{Y3}-S—R^{Y3}, -L^{Y3}-S(═O)—R^{Y3}, or -L^{Y3}-S(═O)₂—R^{Y3},
wherein:
-L^{Y3}- is independently saturated aliphatic C₁₋₄alkylene, and
—R^{Y3} is independently saturated aliphatic C₁₋₄alkyl, saturated C₃₋₆cycloalkyl, C₅₋₆heteroaryl, —CH₂—C₅₋₆heteroaryl, -Ph, or —CH₂-Ph,
wherein each of said C₃₋₆cycloalkyl, C₅₋₆heteroaryl, and -Ph is optionally substituted.

In some embodiments, each of said C₃₋₆cycloalkyl, C₅₋₆heteroaryl, and -Ph is optionally substituted, for example, with one or more groups independently selected from:
—F, —Cl, —Br, —I,
—R^{Y3A}, —CF₃,
—OH, —OR^{Y3A}, —OCF₃,
—SR^{Y3A},
—NH₂, —NHR^{Y3A}, —NR^{Y3A}₂, pyrrolidino, piperidino, morpholino, piperizino, (N—C₁₋₄alkyl)-piperizino,
—C(═O)OH, —C(═O)OR^{Y3A},
—C(═O)R^{Y3A},
—OC(═O)R^{Y3A},
—C(═O)NH₂, —C(═O)NHR^{Y3A}, —C(═O)NR^{Y3A}₂, —C(═O)-pyrrolidino, —C(═O)-piperidino, —C(═O)-morpholino, —C(═O)-piperizino, —C(═O)—(N—C₁₋₄alkyl)-piperizino,
—NHC(═O)R^{Y3A}, —NR^{Y3A}C(═O)R^{Y3A},
—OC(═O)NH₂, —OC(═O)NHR^{Y3A}, —OC(═O)NR^{Y3A}₂, —OC(═O)-pyrrolidino, —OC(═O)-piperidino, —OC(═O)-morpholino, —OC(═O)-piperizino, (N—C₁₋₄alkyl)-piperizino,
—NHC(═O)OH, —NHC(═O)OR^{Y3A}, —NR^{Y3A}C(═O)OR^{Y3A},
—NHC(═O)NH₂, —NHC(═O)NHR^{Y3A}, —NHC(═O)NR^{Y3A}₂, —NHC(═O)-pyrrolidino, —NHC(═O)-piperidino, —NHC(═O)-morpholino, —NHC(═O)-piperizino, —NHC(═O)—(N—C₁₋₄alkyl)-piperizino,
—NO₂, and —CN,
wherein each —R^{Y3A} is independently saturated aliphatic C₁₋₄alkyl, saturated C₃₋₆cycloalkyl, -Ph, or —CH₂-Ph, wherein each of said C₃₋₆cycloalkyl and -Ph is optionally substituted with one or more substituents selected from —F, —Cl, —Br, —I, —R^{Y3B}, —CF₃, —OH, —OR^{Y3B}, and —OCF₃, wherein each —R^{Y3B} is independently saturated aliphatic C₁₋₄alkyl.

In some embodiments -B³ is independently -L^{Y3}-SH or -L^{Y3}-S—R^{Y3}.

In some embodiments -B³ is independently -L^{Y3}-S—R^{Y3}.

In some embodiments -B³ is independently -L^{Y3}-S(═O)—R^{Y3} or -L^{Y3}-S(═O)₂—R^{Y3}.

In some embodiments -L^{Y3}- is independently —CH₂—, —CH₂CH₂—, —CH₂CH₂CH₂—, —CH(CH₃)—, —CH(CH₃)CH₂—, —CH₂CH(CH₃)—, or —CH(CH₂CH₃)—.

In some embodiments -L^{Y3}- is independently —CH₂—, —CH₂CH₂—, or —CH₂CH₂CH₂—.

In some embodiments -L^{Y3}- is independently —CH₂— or —CH₂CH₂—.

In some embodiments —R^{Y3}, if present, is independently saturated aliphatic C₁₋₄alkyl, -Ph, or —CH₂-Ph, wherein said -Ph is optionally substituted.

In some embodiments —R^{Y3}, if present, is independently saturated aliphatic C₁₋₄alkyl.

In some embodiments —R^{Y3}, if present, is independently -Me, -Et, -nPr, -iPr, -nBu, -sBu, -iBu, or -tBu.

### B⁴—3-Oxygen-Alkyl Groups

In some embodiments, -B⁸ is independently -B⁴, wherein -B⁴ is independently hydroxy-C₁₋₄alkyl or ether-C₁₋₄alkyl.

In some embodiments, -B⁴ is independently:
-L^{Y4}-OH or -L^{Y4}-O—R^{Y4},
wherein:
-L^{Y4}- is independently saturated aliphatic C₁₋₄alkylene, and
—R^{Y4} is independently saturated aliphatic C₁₋₄alkyl, saturated C₃₋₆cycloalkyl, C₅₋₆heteroaryl, —CH₂—C₅₋₆heteroaryl, -Ph, or —CH₂-Ph,
wherein each of said C₃₋₆cycloalkyl, C₅₋₆heteroaryl, and -Ph is optionally substituted.

In some embodiments, each of said C₃₋₆cycloalkyl, C₅₋₆heteroaryl, and -Ph is optionally substituted with one or more groups independently selected from:
—F, —Cl, —Br, —I,
—R^{Y4A}, —CF₃,
—OH, —OR^{Y4A}, —OCF₃,
—SR^{Y4A},
—NH₂, —NHR^{Y4A}, —NR^{Y4A}₂, pyrrolidino, piperidino, morpholino, piperizino, (N—C₁₋₄alkyl)-piperizino,
—C(═O)OH, —C(═O)OR^{Y4A},
—C(═O)R^{Y4A},
—OC(═O)R^{Y4A},
—C(═O)NH₂, —C(═O)NHR^{Y4A}, —C(═O)NR^{Y4A}₂, —C(═O)-pyrrolidino, —C(═O)-piperidino, —C(═O)-morpholino, —C(═O)-piperizino, —C(═O)—(N—C₁₋₄alkyl)-piperizino,
—NHC(═O)R^{Y4A}, —NR^{Y4A}C(═O)R^{Y4A},
—OC(═O)NH₂, —OC(═O)NHR^{Y4A}, —OC(═O)NR^{Y4A}₂, —OC(═O)-pyrrolidino, —OC(═O)-piperidino, —OC(═O)-morpholino, —OC(═O)-piperizino, (N—C₁₋₄alkyl)-piperizino,
—NHC(═O)OH, —NHC(═O)OR^{Y4A}, —NR^{Y4A}C(═O)OR^{Y4A},
—NHC(═O)NH₂, —NHC(═O)NHR^{Y4A}, —NHC(═O)NR^{Y4A}₂, —NHC(═O)-pyrrolidino, —NHC(═O)-piperidino, —NHC(═O)-morpholino, —NHC(═O)-piperizino, —NHC(═O)—(N—C₁₋₄alkyl)-piperizino,
—NO₂, and —CN,
wherein each —R^{Y4A} is independently saturated aliphatic C₁₋₄alkyl, saturated C₃₋₆cycloalkyl, -Ph, or —CH₂-Ph, wherein each of said C₃₋₆cycloalkyl and -Ph is optionally substituted with one or more substituents selected from —F, —Cl, —Br, —I, —R^{Y4B}, —CF₃, —OH, —OR^{Y4B}, and —OCF₃, wherein each —R^{Y4B} is independently saturated aliphatic C₁₋₄alkyl.

In some embodiments -B⁴ is independently -L^{Y4}-OH or -L^{Y4}-O—R^{Y4}.

In some embodiments -B⁴ is independently -L^{Y4}-O—R^{Y4}.

In some embodiments -L^{Y4}- is independently —CH₂—, —CH₂CH₂—, —CH₂CH₂CH₂—, —CH(CH₃)—, —CH(CH₃)CH₂—, —CH₂CH(CH₃)—, or —CH(CH₂CH₃)—.

In some embodiments -L^{Y4}- is independently —CH₂—, —CH₂CH₂—, or —CH₂CH₂CH₂—.

In some embodiments -L^{Y4}- is independently —CH₂— or —CH₂CH₂—.

In some embodiments —R^{Y4}, if present, is independently saturated aliphatic C₁₋₄alkyl, -Ph, or —CH₂-Ph, wherein said -Ph is optionally substituted.

In some embodiments —R^{Y4}, if present, is independently saturated aliphatic C₁₋₄alkyl.

In some embodiments —R^{Y4}, if present, is independently -Me, -Et, -nPr, -iPr, -nBu, -sBu, -iBu, or -tBu.

In some embodiments, -B⁴ is independently —CH₂—OH.

### B⁵—3-Aryl-Alkyl Groups

In some embodiments, -B^{A} is -B⁵, wherein -B⁵ is independently phenyl-C₁₋₆alkyl or C₅₋₆heteroaryl-C₁₋₆alkyl, and is optionally substituted.

In some embodiments -B⁵ is independently -L^{Y5}-Ar^{Y5}, wherein:
-L^{Y5}- is independently saturated aliphatic C₁₋₄alkylene, and
—Ar^{Y5} is independently C₅₋₆heteroaryl or -Ph,
wherein each of said C₅₋₆heteroaryl and -Ph is optionally substituted.

In some embodiments, each of said C₅₋₆heteroaryl and -Ph is optionally substituted with one or more groups independently selected from:
—F, —Cl, —Br, —I,
—R^{Y5A}, —CF₃,
—OH, —OR^{Y5A}, —OCF₃,
—SR^{Y5A},
—NH₂, —NHR^{Y5A}, —NR^{Y5A}₂, pyrrolidino, piperidino, morpholino, piperizino, (N—C₁₋₄alkyl)-piperizino,
—C(═O)OH, —C(═O)OR^{Y5A},
—C(═O)R^{Y5A},
—OC(═O)R^{Y5A},
—C(═O)NH₂, —C(═O)NHR^{Y5A}, —C(═O)NR^{Y5A}₂, —C(═O)-pyrrolidino, —C(═O)-piperidino, —C(═O)-morpholino, —C(═O)-piperizino, —C(═O)—(N—C₁₋₄alkyl)-piperizino,
—NHC(═O)R^{Y5A}, —NR^{Y5A}C(═O)R^{Y5A},
—OC(═O)NH₂, —OC(═O)NHR^{Y5A}, —OC(═O)NR^{Y5A}₂, —OC(═O)-pyrrolidino, —OC(═O)-piperidino, —OC(═O)-morpholino, —OC(═O)-piperizino, (N—C₁₋₄alkyl)-piperizino,
—NHC(═O)OH, —NHC(═O)OR^{Y5A}, —NR^{Y5A}C(═O)OR^{Y5A},
   —NHC(═O)NH₂, —NHC(═O)NHR^{Y5A}, —NHC(═O)NR^{Y5A}₂, —NHC(═O)-pyrrolidino, —NHC(═O)-piperidino, —NHC(═O)-morpholino, —NHC(═O)-piperizino, —NHC(═O)—(N—C₁₋₄alkyl)-piperizino,
—NO₂, and —CN,
wherein each —R^{Y5A} is independently saturated aliphatic C₁₋₄alkyl, saturated C₃₋₆cycloalkyl, -Ph, or —CH₂-Ph, wherein each of said C₃₋₆cycloalkyl and -Ph is optionally substituted with one or more substituents selected from —F, —Cl, —Br, —I, —R^{Y58}, —CF₃, —OH, —OR^{Y5B}, and —OCF₃, wherein each —R^{Y5B} is independently saturated aliphatic C₁₋₄alkyl.

In some embodiments -L^{Y5}- is independently —CH₂—, —CH₂CH₂—, —CH₂CH₂CH₂—, —CH(CH₃)—, —CH(CH₃)CH₂—, —CH₂CH(CH₃)—, or —CH(CH₂CH₃)—.

In some embodiments —Ar^{Y5} is independently furanyl, thienyl, pyrrolyl, imidazolyl, pyrazolyl, triazolyl, tetrazolyl, oxazolyl, isoxazolyl, thiazolyl, isothiazolyl, pyridyl, pyrimidinyl, pyridazinyl, or -Ph,
wherein each of said furanyl, thienyl, pyrrolyl, imidazolyl, pyrazolyl, triazolyl, tetrazolyl, oxazolyl, isoxazolyl, thiazolyl, isothiazolyl, pyridyl, pyrimidinyl, pyridazinyl, and -Ph is optionally substituted.

In some embodiments —Ar^{Y5} is independently C₅₋₆heteroaryl, or -Ph, wherein each of said C₅₋₆heteroaryl and -Ph is optionally substituted with one or more groups selected from: —F, —Cl, —Br, —I, —R^{Y5A}, —CF₃, —OH, —OR^{Y5A}, and —OCF₃.

In some embodiments —Ar^{Y5} is independently -Ph, wherein said -Ph is optionally substituted with one or more groups selected from: —F, —Cl, —Br, —I, —R^{Y5A}, —CF₃, —OH, —OR^{Y5A}, and —OCF₃.

In some embodiments -B⁵ is —CH₂-Ph.

### B⁶—8-Acyl-Alkyl, 8-Acid-Alkyl, and 8-Ester-Alkyl Groups

In some embodiments, -B^{A} is -B⁶, wherein -B⁶ is independently acyl-C₁₋₆alkyl, carboxy-C₁₋₆alkyl, oxyacyl-C₁₋₆alkyl, or acyloxy-C₁₋₆alkyl.

In some embodiments, -B⁶ is independently:
-L^{Y6}-C(═O)R^{Y6}, -L^{Y6}-C(═O)OH, -L^{Y6}-C(═O)OR^{Y6}, or -L^{Y6}-O—C(═O)R^{Y6},
wherein:
-L^{Y6}- is independently saturated aliphatic C₁₋₄alkylene, and
—R^{Y6} is independently saturated aliphatic C₁₋₄alkyl, saturated C₃₋₆cycloalkyl, C₅₋₆heteroaryl, —CH₂—C₅₋₆heteroaryl, -Ph, or —CH₂-Ph,
wherein each of said C₃₋₆cycloalkyl, C₅₋₆heteroaryl, and -Ph is optionally substituted.

In some embodiments, each of said C₃₋₆cycloalkyl, C₅₋₆heteroaryl, and -Ph is optionally substituted, for example, with one or more groups independently selected from:
—F, —Cl, —Br, —I,
—R^{Y6A}, —CF₃,
—OH, —OR^{Y6A}, —OCF₃,
—SR^{Y6A},
—NH₂, —NHR^{Y6A}, —NR^{Y6A}₂, pyrrolidino, piperidino, morpholino, piperizino, (N—C₁₋₄alkyl)-piperizino,
—C(═O)OH, —C(═O)OR^{Y6A},
—C(═O)R^{Y6A},
—OC(═O)R^{Y6A},
—C(═O)NH₂, —C(═O)NHR^{Y6A}, —C(═O)NR^{Y6A}₂, —C(═O)-pyrrolidino, —C(═O)-piperidino, —C(═O)-morpholino, —C(═O)-piperizino, —C(═O)—(N—C₁₋₄alkyl)-piperizino,
—NHC(═O)R^{Y6A}, —NR^{Y6A}C(═O)R^{Y6A},
—OC(═O)NH₂, —OC(═O)NHR^{Y6A}, —OC(═O)NR^{Y6A}₂, —OC(═O)-pyrrolidino, —OC(═O)-piperidino, —OC(═O)-morpholino, —OC(═O)-piperizino, (N—C₁₋₄alkyl)-piperizino,
—NHC(═O)OH, —NHC(═O)OR^{Y6A}, —NR^{Y6A}C(═O)OR^{Y6A},
—NHC(═O)NH₂, —NHC(═O)NHR^{Y6A}, —NHC(═O)NR^{Y6A}₂, —NHC(═O)-pyrrolidino, —NHC(═O)-piperidino, —NHC(═O)-morpholino, —NHC(═O)-piperizino, —NHC(═O)—(N—C₁₋₄alkyl)-piperizino,
—NO₂, and —CN,
wherein each —R^{Y6A} is independently saturated aliphatic C₁₋₄alkyl, saturated C₃₋₆cycloalkyl, -Ph, or —CH₂-Ph, wherein each of said C₃₋₆cycloalkyl and -Ph is optionally substituted with one or more substituents selected from —F, —Cl, —Br, —I, —R^{Y6B}, —CF₃, —OH, —OR^{Y6B}, and —OCF₃, wherein each —R^{Y6B} is independently saturated aliphatic C₁₋₄alkyl.

In some embodiments -B⁶ is independently -L^{Y6}-C(═O)R^{Y6}, -L^{Y6}-C(═O)OH, -L^{Y6}-C(═O)OR^{Y6}, or -L^{Y6}-O—C(═O)R^{Y6}.

In some embodiments -L^{Y6}- is independently —CH₂—, —CH₂CH₂—, —CH₂CH₂CH₂—, —CH(CH₃)—, —CH(CH₃)CH₂—, —CH₂CH(CH₃)—, or —CH(CH₂CH₃)—.

In some embodiments —R^{Y6}, if present, is independently saturated aliphatic C₁₋₄alkyl, -Ph, or —CH₂-Ph, wherein said -Ph is optionally substituted.

In some embodiments —R^{Y6}, if present, is independently saturated aliphatic C₁₋₄alkyl.

In some embodiments —R^{Y6}, if present, is independently -Me, -Et, -nPr, -iPr, -nBu, -sBu, -iBu, or -tBu.

In some embodiments -B⁶ is independently —CH₂—C(═O)—O-Et.

### B⁷—3-Amido-Alkyl Groups

In some embodiments, -B is -B⁷, wherein -B⁷ is independently amido-C₁₋₄alkyl or substituted amido-C₁₋₄alkyl.

In some embodiments, -B⁷ is independently:
-L^{Y7}-C(═O)NH₂, -L^{Y7}-C(═O)NHR^{Y7}, -L^{Y7}-C(═O)NR^{Y7}₂, -L^{Y7}-C(═O)-pyrrolidino, -L^{Y7}-C(═O)-piperidino, -L^{Y7}-C(═O)-morpholino, -L^{Y7}-C(═O)-piperizino, or -L^{Y7}-C(═O)—(N—C₁₋₄alkyl)-piperizino,
wherein:
-L^{Y7}- is independently saturated aliphatic C₁₋₄alkylene, and
—R^{Y7} is independently saturated aliphatic C₁₋₄alkyl, saturated C₃₋₆cycloalkyl, C₅₋₆heteroaryl, —CH₂—C₅₋₆heteroaryl, -Ph, or —CH₂-Ph,
wherein each of said C₃₋₆cycloalkyl, C₅₋₆heteroaryl, and -Ph is optionally substituted.

In some embodiments, each of said C₃₋₆cycloalkyl, C₅₋₆heteroaryl, and -Ph is optionally substituted, for example, with one or more groups independently selected from:
—F, —Cl, —Br, —I,
—R^{Y7A}, —CF₃,
—OH, —OR^{Y7A}, —OCF₃,
—SR^{Y7A},
—NH₂, —NHR^{Y7A}, —NR^{Y7A}₂, pyrrolidino, piperidino, morpholino, piperizino, (N—C₁₋₄alkyl)-piperizino,
—C(═O)OH, —C(═O)OR^{Y7A},
—C(═O)R^{Y7A},
—OC(═O)R^{Y7A},
—C(═O)NH₂, —C(═O)NHR^{Y7A}, —C(═O)NR^{Y7A}₂, —C(═O)-pyrrolidino, —C(═O)-piperidino, —C(═O)-morpholino, —C(═O)-piperizino, —C(═O)—(N—C₁₋₄alkyl)-piperizino,
—NHC(═O)R^{Y7A}, —NR^{Y7A}C(═O)R^{Y7A},
—C(═O)NH₂, —OC(═O)NHR^{Y7A}, —OC(═O)NR^{Y7A}₂, —OC(═O)-pyrrolidino, —OC(═O)-piperidino, —OC(═O)-morpholino, —OC(═O)-piperizino, (N—C₁₋₄alkyl)-piperizino,
—NHC(═O)OH, —NHC(═O)OR^{Y7A}, —NR^{Y7A}C(═O)OR^{Y7A},
—NHC(═O)NH₂, —NHC(═O)NHR^{Y7A}, —NHC(═O)NR^{Y7A}₂, —NHC(═O)-pyrrolidino, —NHC(═O)-piperidino, —NHC(═O)-morpholino, —NHC(═O)-piperizino, —NHC(═O)—(N—C₁₋₄alkyl)-piperizino,
—NO₂, and —CN,
wherein each —R^{Y7A} is independently saturated aliphatic C₁₋₄alkyl, saturated C₃₋₆cycloalkyl, -Ph, or —CH₂-Ph, wherein each of said C₃₋₆cycloalkyl and -Ph is optionally substituted with one or more substituents selected from —F, —Cl, —Br, —I, —R^{Y7B}, —CF₃, —OH, —OR^{Y78}, and —OCF₃, wherein each —R^{Y7B} is independently saturated aliphatic C₁₋₄alkyl.

In some embodiments, -B⁷ is independently:
-L^{Y7}-C(═O)NH₂, -L^{Y7}-C(═O)NHR^{Y7}, -L^{Y7}-C(═O)NR^{Y7}₂.

In some embodiments -B⁷ is independently -L^{Y7}-C(═O)NH₂.

In some embodiments -L^{Y7}- is independently —CH₂—, —CH₂CH₂—, —CH₂CH₂CH₂—, —CH(CH₃)—, —CH(CH₃)CH₂—, —CH₂CH(CH₃)—, or —CH(CH₂CH₃)—.

In some embodiments —R^{Y7}, if present, is independently saturated aliphatic C₁₋₄alkyl, -Ph, or —CH₂-Ph, wherein said -Ph is optionally substituted.

In some embodiments -B⁷ is independently:
—CH₂—C(═O)NH₂, —CH₂—C(═O)NHMe, —CH₂—C(═O)NMe₂,
—CH₂CH₂—C(═O)NH₂, —CH₂CH₂—C(═O)NHMe, —CH₂CH₂—C(═O)NMe₂,
—CH₂—C(═O)-piperidino, or —CH₂CH₂—C(═O)-piperidino.

### B⁸—3-Cyclic and 3-Cyclic-Alkyl Groups

In some embodiments, -B is -B⁸, wherein -B⁸ is independently C₃₋₆cycloalkyl, C₃₋₆cycloalkyl-C₁₋₄alkyl, C₃₋₆heterocyclyl, or C₃₋₆heterocyclyl-C₁₋₄alkyl, and is optionally substituted.

In some embodiments, -B⁸ is independently:
—R^{Y8} or -L^{Y8}-R^{Y8},
wherein:
-L^{Y8}- is independently saturated aliphatic C₁₋₄alkylene, and
—R^{Y8} is independently saturated C₃₋₆cycloalkyl or saturated C₃₋₆heterocyclyl,
wherein each of said C₃₋₆cycloalkyl and C₃₋₆heterocyclyl is optionally substituted.

In some embodiments, each of said C₃₋₆cycloalkyl and C₃₋₆heterocyclyl is optionally substituted, for example, with one or more groups selected from:
—F, —Cl, —Br, —I,
—R^{Y8A}, —CF₃,
—OH, —OR^{Y8A}, —OCF₃,
—NH₂, —NHR^{Y8A}, —NR^{Y8A}₂, pyrrolidino piperidino, morpholino, piperizino, (N—C₁₋₄alkyl)-piperizino,
—C(═O)OH, —C(═O)OR^{Y8A},
—C(═O)R^{Y8A},
—OC(═O)R^{Y8A},
—C(═O)NH₂, —C(═O)NHR^{Y8A}, —C(═O)NR^{Y8A}₂, —C(═O)-pyrrolidino, —C(═O)-piperidino, —C(═O)-morpholino, —C(═O)-piperizino, —C(═O)—(N—C₁₋₄alkyl)-piperizino,
—NHC(═O)R^{Y8A}, —NR^{Y8A}C(═O)R^{Y8A}, and
—CN;
wherein each —R^{Y8A} is independently saturated aliphatic C₁₋₄alkyl, saturated C₃₋₆cycloalkyl, -Ph, or —CH₂-Ph, wherein each of said C₃₋₆cycloalkyl and -Ph is optionally substituted with one or more substituents selected from —F, —Cl, —Br, —I, —R^{Y8B}, —CF₃, —OH, —OR^{Y8B}, and —OCF₃, wherein each —R^{Y8B} is independently saturated aliphatic C₁₋₄alkyl.

In some embodiments -L^{Y8}-, if present, is independently —CH₂—, —CH₂CH₂—, —CH₂CH₂CH₂—, —CH(CH₃)—, —CH(CH₃)CH₂—, —CH₂CH(CH₃)—, or —CH(CH₂CH₃)—.

In some embodiments -L^{Y8}-, if present, is independently —CH₂—.

In some embodiments —R^{Y8} is independently saturated C₃₋₆cycloalkyl, and is optionally substituted.

In some embodiments —R^{Y8} is cyclopropyl.

In some embodiments, —R^{Y8} is independently saturated C₃₋₆heterocyclyl, and is optionally substituted.

In some embodiments —R^{Y8} is independently saturated pyrrolidinyl, piperidinyl, piperizinyl, or morpholinyl, and is optionally substituted.

In some embodiments —R^{Y8} is independently epoxide, oxetane, tetrahydrofuran, or tetrahydropyran, and is optionally substituted.

In some embodiments —R^{Y8} is independently epoxide.

In some embodiments, -B⁸ is —CH₂-epoxide.

### B⁹—8-Halo-Alkyl Groups

In some embodiments, -B is -B⁹, wherein -B⁹ is independently halo-C₁₋₆alkyl.

As used herein, the term “haloalkyl” relates to a saturated aliphatic alkyl group in which one or more hydrogen atoms has been replaced with a halogen atom selected from —F, —Cl, —Br, and —I.

In some embodiments, -B⁹ is independently halo-C₁₋₄alkyl.

In some embodiments, -B⁹ is independently selected from:
—CH₂F, —CH₂CH₂F, —CH₂CH₂CH₂F,
—CH₂CH₂Cl, —CH₂CH₂CH₂Cl,
—CH₂Br, —CH₂CH₂Br, —CH₂CH₂CH₂Br,
—CH₂I, —CH₂CH₂I, —CH₂CH₂CH₂I,
CHF₂, —CH₂CHF₂, —CH₂CH₂CHF₂,
—CF₃, —CH₂CF₃, and —CH₂CH₂CF₃.

In some embodiments, -B⁹ is independently selected from —CH₂CH₂F, —CH₂CHF₂, and —CH₂CF₃.

### B¹⁰—8-Nitro-Alkyl Groups

In some embodiments, -B is -B¹⁰, wherein -B¹⁰ is independently nitro-C₁₋₆alkyl.

In some embodiments, -B¹⁰ is independently -L^{Y10}-NO₂, wherein -L^{Y10}- is independently saturated aliphatic C₁₋₄alkylene.

In some embodiments -L^{Y10}- is independently —CH₂—, —CH₂CH₂—, —CH₂CH₂CH₂—, —CH(CH₃)—, —CH(CH₃)CH₂—, —CH₂CH(CH₃)—, or —CH(CH₂CH₃)—.

In some embodiments -L^{Y10}- is independently —CH₂—.

(In some embodiments -B¹⁰ is independently —CH₂—NO₂.

### B¹¹—8-Cyano-Alkyl Groups

In some embodiments, -B is -B¹¹ wherein -B¹¹ is independently cyano-C₁₋₆alkyl.

In some embodiments, -B¹¹ is independently -L^{Y11}-CN, wherein -L^{Y11}- is independently saturated aliphatic C₁₋₄alkylene.

In some embodiments, -L^{Y11}- is independently —CH₂—, —CH₂CH₂—, —CH₂CH₂CH₂—, —CH(CH₃)—, —CH(CH₃)CH₂—, —CH₂CH(CH₃)—, or —CH(CH₂CH₃)—.

In some embodiments -L^{Y11}- is independently —CH₂— or —CH₂CH₂—.

In some embodiments -B¹¹ is independently —CH₂—CN.

### B¹²—8-Phosphate-Alkyl Groups

In some embodiments, -B is -B¹², wherein -B¹² is independently phosphate-C₁₋₆alkyl.

In some embodiments, -B¹² is independently:
-L^{Y12}-P(═O)(OH)₂, -L^{Y12}-P(═O)(OH)(OR^{Y12}), or -L^{Y12}-P(═O)(OR^{Y12})₂,
wherein:
-L^{Y12}- is independently saturated aliphatic C₁₋₄alkylene, and
each —R^{Y12} is independently saturated aliphatic C₁₋₄alkyl, saturated C₃₋₆cycloalkyl, C₅₋₆heteroaryl, —CH₂—C₅₋₆heteroaryl, -Ph, or —CH₂-Ph,
wherein each of said C₃₋₆cycloalkyl, C₅₋₆heteroaryl, and -Ph is optionally substituted.

In some embodiments, each of said C₃₋₆cycloalkyl, C₅₋₆heteroaryl, and -Ph is optionally substituted, for example, with one or more groups selected from:
—F, —Cl, —Br, —I,
—R^{Y12A}, —CF₃,
—OH, —OR^{Y12A}, —OCF₃,
—NH₂, —NHR^{Y12A}, —NR^{Y12A}₂, pyrrolidino piperidino, morpholino, piperizino, (N—C₁₋₄alkyl)-piperizino,
—C(═O)OH, —C(═O)OR^{Y12A},
—C(═O)R^{Y12A},
—OC(═O)R^{Y12A},
—C(═O)NH₂, —C(═O)NHR^{Y12A}, —C(═O)NR^{Y12A}₂, —C(═O)-pyrrolidino, —C(═O)-piperidino, —C(═O)-morpholino, —C(═O)-piperizino, —C(═O)—(N—C₁₋₄alkyl)-piperizino,
—NHC(═O)R^{Y12A}, —NR^{Y12A}C(═O)R^{Y12A}, and
—CN;
wherein each —R^{Y12A} is independently saturated aliphatic C₁₋₄alkyl, saturated C₃₋₆cycloalkyl, -Ph, or —CH₂-Ph, wherein each of said C₃₋₆cycloalkyl and -Ph is optionally substituted with one or more substituents selected from —F, —Cl, —Br, —I, —R^{Y12B}, —CF₃, —OH, —OR^{Y12B}, and —OCF₃, wherein each —R^{Y12B} is independently saturated aliphatic C₁₋₄alkyl.

In some embodiments -B¹² is independently -L^{Y12}-P(═O)(OH)₂.

In some embodiments -B¹² is independently -L^{Y12}-P(═O)(OH)(OR^{Y12}).

In some embodiments -B¹² is independently -L^{Y12}-P(═O)(OR^{Y12})₂.

In some embodiments -L^{Y12}- is independently —CH₂—, —CH₂CH₂—, —CH₂CH₂CH₂—, —CH(CH₃)—, —CH(CH₃)CH₂—, —CH₂CH(CH₃)—, or —CH(CH₂CH₃)—.

In some embodiments, each —R^{Y12}, if present, is independently saturated aliphatic C₁₋₄alkyl, -Ph, or —CH₂-Ph, wherein said -Ph is optionally substituted.

In some embodiments each —R^{Y12}, if present, is independently saturated aliphatic C₁₋₄alkyl.

In some embodiments each —R^{Y12}, if present, is independently -Me, -Et, -nPr, -iPr, -nBu, -sBu, -iBu, or -tBu.

In some embodiments each —R^{Y12}, if present, is independently -Me, -Et, -nPr, or -iPr.

In some embodiments each —R^{Y12}, if present, is independently -Me or -Et.

In some embodiments each —R^{Y12}, if present, is independently -Et.

In some embodiments each -B¹² is —CH₂—P(═O)(OEt)₂.

### B¹³—8-Carbamate-Alkyl Groups

In some embodiments, -B is -B¹³, wherein -B¹³ is independently carbamate-C₁₋₆alkyl.

In some embodiments, -B¹³ is independently:
-L^{Y13}-NH—C(═O)OH, -L^{Y13}-NH—C(═O)—R^{Y13}, -L^{Y13}-NR^{Y13}—C(═O)OH, or -L^{Y13}-NR^{Y13}—C(═O)—R^{Y13},
wherein:
-L^{Y13}- is independently saturated aliphatic C₁₋₄alkylene, and
each —R^{Y13} is independently saturated aliphatic C₁₋₄alkyl, saturated C₃₋₆cycloalkyl, C₅₋₆heteroaryl, —CH₂—C₅₋₆heteroaryl, fluorenyl, —CH₂-fluorenyl, -Ph, or —CH₂-Ph,
wherein each of said C₃₋₆cycloalkyl, C₅₋₆heteroaryl, fluorenyl and -Ph is optionally substituted.

In some embodiments, each of said C₃₋₆cycloalkyl, C₅₋₆heteroaryl, fluorenyl and -Ph is optionally substituted, for example, with one or more groups selected from:
—F, —Cl, —Br, —I,
—R^{Y13A}, —CF₃,
—OH, —OR^{Y13A}, —OCF₃,
—NH₂, —NHR^{Y13A}, —NR^{Y13A}₂, pyrrolidino piperidino, morpholino, piperizino, (N—C₁₋₄alkyl)-piperizino,
—C(═O)OH, —C(═O)OR^{Y13A},
—C(═O)R^{Y13A},
—OC(═O)R^{Y13A},
—C(═O)NH₂, —C(═O)NHR^{Y13A}, —C(═O)NR^{Y13A}₂, —C(═O)-pyrrolidino, —C(═O)-piperidino, —C(═O)-morpholino, —C(═O)-piperizino, —C(═O)—(N—C₁₋₄alkyl)-piperizino,
—NHC(═O)R^{Y13A}, —NR^{Y13A}C(═O)R^{Y13A}, and
—CN;
wherein each —R^{Y13A} is independently saturated aliphatic C₁₋₄alkyl, saturated C₃₋₆cycloalkyl, -Ph, or —CH₂-Ph, wherein each of said C₃₋₆cycloalkyl and -Ph is optionally substituted with one or more substituents selected from —F, —Cl, —Br, —I, —R^{Y13B}, —CF₃, —OH, —OR^{Y13B}, and —OCF₃, wherein each —R^{Y13B} is independently saturated aliphatic C₁₋₄alkyl.

In some embodiments -L^{Y13}- is independently —CH₂—, —CH₂CH₂—, —CH₂CH₂CH₂—, —CH(CH₃)—, —CH(CH₃)CH₂—, —CH₂CH(CH₃)—, or —CH(CH₂CH₃)—.

In some embodiments each —R^{Y13}, if present, is independently saturated aliphatic C₁₋₄alkyl, saturated C₃₋₆cycloalkyl, fluorenyl, —CH₂-fluorenyl, -Ph, or —CH₂-Ph, wherein each of said C₃₋₆cycloalkyl, C₅₋₆heteroaryl, fluorenyl and -Ph is optionally substituted with one or more groups selected from: —F, —Cl, —Br, —I, —R^{Y12A}, —CF₃, —OH, —OR^{Y12A}, and —OCF₃.

In some embodiments, —R^{Y13} is fluorenyl or —CH₂-fluorenyl.

### B¹⁴—8-Oxime-Alkyl Groups

In some embodiments, -B is -B¹⁴, wherein -B¹⁴ is independently oxime-C₁₋₆alkyl.

In some embodiments, -B¹⁴ is independently:
-L^{Y14}-CH(═N—O—H), -L^{Y14}-CH(═N—O—R^{Y14}), -L^{Y14}-CR^{Y14}(═N—O—H), or -L^{Y14}-CR^{Y14}(═N—O—R^{Y14}),
wherein:
-L^{Y14}- is independently saturated aliphatic C₁₋₄alkylene, and
each —R^{Y14} is independently saturated aliphatic C₁₋₄alkyl, saturated C₃₋₆cycloalkyl, C₅₋₆heteroaryl, —CH₂—C₅₋₆heteroaryl, -Ph, or —CH₂-Ph,
wherein each of said C₃₋₆cycloalkyl, C₅₋₆heteroaryl, and -Ph is optionally substituted.

In some embodiments, each of said C₃₋₆cycloalkyl, C₅₋₆heteroaryl, and -Ph is optionally substituted with one or more groups selected from:
—F, —Cl, —Br, —I,
—R^{Y14A}, —CF₃,
—OH, —OR^{Y14A}, —OCF₃,
—NH₂, —NHR^{Y14A}, —NR^{Y14A}₂, pyrrolidino piperidino, morpholino, piperizino, (N—C₁₋₄alkyl)-piperizino,
—C(═O)OH, —C(═O)OR^{Y14A},
—C(═O)R^{Y14A},
—OC(═O)R^{Y14A},
—C(═O)NH₂, —C(═O)NHR^{Y14A}, —C(═O)NR^{Y14A}₂, —C(═O)-pyrrolidino, —C(═O)-piperidino, —C(═O)-morpholino, —C(═O)-piperizino, —C(═O)—(N—C₁₋₄alkyl)-piperizino,
—NHC(═O)R^{Y14A}, —NR^{Y14A}C(═O)R^{Y14A}, and
—CN;
wherein each —R^{Y14A} is independently saturated aliphatic C₁₋₄alkyl, saturated C₃₋₆cycloalkyl, -Ph, or —CH₂-Ph, wherein each of said C₃₋₆cycloalkyl and -Ph is optionally substituted with one or more substituents selected from —F, —Cl, —Br, —I, —R^{Y14B}, —CF₃, —OH, —OR^{Y14B}, and —OCF₃, wherein each —R^{Y14B} is independently saturated aliphatic C₁₋₄alkyl.

In some embodiments -B¹⁴ is independently -L^{Y14}-CH(═N—O—R^{Y14}) or -L^{Y5}-CR^{Y5}(═N—O—R^{Y5}).

In some embodiments -B¹⁴ is independently -L^{Y14}-CR^{Y14}(═N—O—R^{Y14}).

In some embodiments -L^{Y14}- is independently —CH₂—, —CH₂CH₂—, —CH₂CH₂CH₂—, —CH(CH₃)—, —CH(CH₃)CH₂—, —CH₂CH(CH₃)—, or —CH(CH₂CH₃)—.

In some embodiments each —R^{Y14}, if present, is independently saturated aliphatic C₁₋₄alkyl, -Ph, or —CH₂-Ph, wherein said -Ph is optionally substituted.

In some embodiments each —R^{Y14}, if present, is independently saturated aliphatic C₁₋₄alkyl.

In some embodiments each —R^{Y4}, if present, is independently -Me, -Et, -nPr, -iPr, -nBu, -sBu, -iBu, or -tBu.

In some embodiments each —R^{Y14}, if present, is independently -Me or -Et.

In some embodiments -B¹⁴ is independently —CH₂—C(Et)(═N—O-Me).

### The Groups J¹ and J²

J¹ and J² are each independently —H or —C₁₋₃ alkyl.

In some embodiments, J¹ and J² are each independently selected from —H, -Me, -Et, -nPr, and -iPr.

In some embodiments, J¹ and J² are each independently selected from —H, -Me and -Et.

In some embodiments, J¹ and J² are each independently selected from —H and -Me.

In some embodiments, one of J¹ and J² is —H and the other is independently C₁₋₃ alkyl.

In some embodiments, one of J¹ and J² is —H and the other is independently selected from -Me, -Et, -nPr, and -iPr.

In some embodiments, one of J¹ and J² is H and the other is independently selected from Me and -Et.

In some embodiments, one of J¹ and J² is H and the other is independently -Me.

In some embodiments, J¹ and J² are each independently —H.

In some embodiments, J¹ and J² are each independently -Me.

### The Groups P¹ and P²

P¹ and P² are each independently H or an amine protecting group or P¹ and P² together form an amine protecting group.

A wide variety of amine protecting groups are widely used and well known in organic synthesis. See, for example, Protective Groups in Organic Synthesis (T. Green and P. Wuts; 4th Edition; John Wiley and Sons, 2006).

In some embodiments, the amine protecting group is an acid-cleavable protecting group.

In some embodiments, P¹ and P² are each independently —H or an amine protecting group.

In some embodiments, one of P¹ and P² is —H and the other is an amine protecting group.

In some embodiments, one of P¹ and P² is —H and the other is an amine protecting group selected from tert-butoxycarbonyl (Boc), acetyl (Ac), tetrahydropyran (THP), trimethyl silyl (TMS), triisopropyl silyl (TIPS), tetra-butyl dimethyl silyl (TBDMS), benzyl (Bn), para-methoxybenzyl (PMB), triphenyl methyl (trityl).

In some embodiments, P¹ and P² are each independently —H.

In some embodiments, P¹ and P² together form an amine protecting group.

In some embodiments, P¹ and P² together form a cyclic imide, for example a phthalimide group:

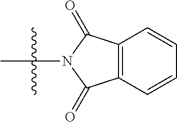 [SMILES: *N1C(=O)c2ccccc2C1=O]

### Isomers

Certain compounds may exist in one or more particular geometric, optical, enantiomeric, diasteriomeric, epimeric, atropic, stereoisomeric, tautomeric, conformational, or anomeric forms, including but not limited to, cis- and trans-forms; E- and Z-forms; c-, t-, and r-forms; endo- and exo-forms; R-, S-, and meso-forms; D- and L-forms; d- and l-forms; (+) and (−) forms; keto-, enol-, and enolate-forms; syn- and anti-forms; synclinal- and anticlinal-forms; α- and p-forms; axial and equatorial forms; boat-, chair-, twist-, envelope-, and halfchair-forms; and combinations thereof, hereinafter collectively referred to as “isomers” (or “isomeric forms”).

Note that, except as discussed below for tautomeric forms, specifically excluded from the term “isomers,” as used herein, are structural (or constitutional) isomers (i.e., isomers which differ in the connections between atoms rather than merely by the position of atoms in space). For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, meta-chlorophenyl. However, a reference to a class of structures may well include structurally isomeric forms falling within that class (e.g., C₁₋₇alkyl includes n-propyl and iso-propyl; butyl includes n-, iso-, sec-, and tert-butyl; methoxyphenyl includes ortho-, meta-, and para-methoxyphenyl).

The above exclusion does not pertain to tautomeric forms, for example, keto-, enol-, and enolate-forms, as in, for example, the following tautomeric pairs: keto/enol (illustrated below), imine/enamine, amide/imino alcohol, amidine/amidine, nitroso/oxime, thioketone/enethiol, N-nitroso/hydroxyazo, and nitro/aci-nitro.

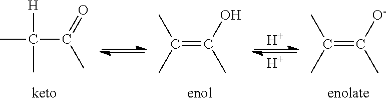

Note that specifically included in the term “isomer” are compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; and the like.

Unless otherwise specified, a reference to a particular compound includes all such isomeric forms, including mixtures (e.g., racemic mixtures) thereof. Methods for the preparation (e.g., asymmetric synthesis) and separation (e.g., fractional crystallisation and chromatographic means) of such isomeric forms are either known in the art or are readily obtained by adapting the methods taught herein, or known methods, in a known manner.

### Salts

It may be convenient or desirable to prepare, purify, and/or handle a corresponding salt of the compound, for example, a pharmaceutically-acceptable salt. Examples of pharmaceutically acceptable salts are discussed in Berge et al., 1977, “Pharmaceutically Acceptable Salts,” J. Pharm. Sci., Vol. 66, pp. 1-19.

For example, if the compound is anionic, or has a functional group which may be anionic (e.g., —COOH may be —COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al⁺³. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

If the compound is cationic, or has a functional group which may be cationic (e.g., —NH₂ may be —NH₃⁺), then a salt may be formed with a suitable anion. Examples of suitable inorganic anions include, but are not limited to, those derived from the following inorganic acids: hydrochloric, hydrobromic, hydroiodic, sulfuric, sulfurous, nitric, nitrous, phosphoric, and phosphorous.

Examples of suitable organic anions include, but are not limited to, those derived from the following organic acids: 2-acetyoxybenzoic, acetic, ascorbic, aspartic, benzoic, camphorsulfonic, cinnamic, citric, edetic, ethanedisulfonic, ethanesulfonic, fumaric, glucheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, toluenesulfonic, and valeric. Examples of suitable polymeric organic anions include, but are not limited to, those derived from the following polymeric acids: tannic acid, carboxymethyl cellulose.

Unless otherwise specified, a reference to a particular compound also includes salt forms thereof.

### Hydrates and Solvates

It may be convenient or desirable to prepare, purify, and/or handle a corresponding hydrate or solvate of the compound (e.g., pharmaceutically acceptable hydrates or solvates of the compound). The term “solvate” is used herein in the conventional sense to refer to a complex of solute (e.g., compound, salt of compound) and solvent. If the solvent is water, the solvate may be conveniently referred to as a hydrate, for example, a mono-hydrate, a di-hydrate, a tri-hydrate, etc.

Unless otherwise specified, a reference to a particular compound also includes hydrate and solvate forms thereof.

### Chemically Protected Forms

It may be convenient or desirable to prepare, purify, and/or handle the compound in a chemically protected form. The term “chemically protected form” is used herein in the conventional chemical sense and pertains to a compound in which one or more reactive functional groups are protected from undesirable chemical reactions under specified conditions (e.g., pH, temperature, radiation, solvent, and the like). In practice, well known chemical methods are employed to reversibly render unreactive a functional group, which otherwise would be reactive, under specified conditions. In a chemically protected form, one or more reactive functional groups are in the form of a protected or protecting group (also known as a masked or masking group or a blocked or blocking group). By protecting a reactive functional group, reactions involving other unprotected reactive functional groups can be performed, without affecting the protected group; the protecting group may be removed, usually in a subsequent step, without substantially affecting the remainder of the molecule. See, for example, Protective Groups in Organic Synthesis (T. Green and P. Wuts; 4th Edition; John Wiley and Sons, 2006).

A wide variety of such “protecting,” “blocking,” or “masking” methods are widely used and well known in organic synthesis. For example, a compound which has two nonequivalent reactive functional groups, both of which would be reactive under specified conditions, may be derivatized to render one of the functional groups “protected,” and therefore unreactive, under the specified conditions; so protected, the compound may be used as a reactant which has effectively only one reactive functional group. After the desired reaction (involving the other functional group) is complete, the protected group may be “deprotected” to return it to its original functionality.

For example, a hydroxy group may be protected as an ether (—OR) or an ester (—OC(═O)R), for example, as: a t-butyl ether; a benzyl, benzhydryl (diphenylmethyl), or trityl (triphenylmethyl)ether; a trimethylsilyl or t-butyldimethylsilyl ether; or an acetyl ester (—OC(═O)CH₃, —OAc).

For example, an aldehyde or ketone group may be protected as an acetal (R—CH(OR)₂) or ketal (R₂C(OR)₂), respectively, in which the carbonyl group (>C═O) is converted to a diether (>C(OR)₂), by reaction with, for example, a primary alcohol. The aldehyde or ketone group is readily regenerated by hydrolysis using a large excess of water in the presence of acid.

For example, an amine group may be protected, for example, as an amide (—NRCO—R) or a urethane (—NRCO—OR), for example, as: a methyl amide (—NHCO—CH₃); a benzyloxy amide (—NHCO—OCH₂C₆H₅, —NH-Cbz); as a t-butoxy amide (—NHCO—OC(CH₃)₃, —NH-Boc); a 2-biphenyl-2-propoxy amide (—NHCO—OC(CH₃)₂C₆H₄C₆H₅, —NH-Bpoc), as a 9-fluorenylmethoxy amide (—NH-Fmoc), as a 6-nitroveratryloxy amide (—NH-Nvoc), as a 2-trimethylsilylethyloxy amide (—NH-Teoc), as a 2,2,2-trichloroethyloxy amide (—NH-Troc), as an allyloxy amide (—NH-Alloc), as a 2(-phenylsulfonyl)ethyloxy amide (—NH-Psec); or, in suitable cases (e.g., cyclic amines), as a nitroxide radical (>N—O.).

For example, a carboxylic acid group may be protected as an ester for example, as: an C₁₋₇alkyl ester (e.g., a methyl ester; a t-butyl ester); a C₁₋₇haloalkyl ester (e.g., a C₁₋₇trihaloalkyl ester); a triC₁₋₇alkylsilyl-C₁₋₇alkyl ester; or a C₅₋₂₀aryl-C₁₋₇alkyl ester (e.g., a benzyl ester; a nitrobenzyl ester); or as an amide, for example, as a methyl amide.

For example, a thiol group may be protected as a thioether (—SR), for example, as: a benzyl thioether; an acetamidomethyl ether (—S—CH₂NHC(═O)CH₃).

For example, a carbonyl group may be protected as an oxime (—C(═NOH)—) or a substituted oxime (—C(═NOR)—), for example, where R is saturated aliphatic C₁₋₄alkyl.

### Examples

The following examples are provided solely to illustrate the present invention and are not intended to limit the scope of the invention, as described herein.

### General

Melting points were recorded on Stuart Scientific SMP3 apparatus, and are uncorrected. IR spectra were recorded on a Shimadzu FT-IR8400S, and NMR spectra were recorded on a Bruker Avance 400 instrument at 400.13 MHz (¹H), 100.62 MHz (¹³C) and 40.54 (¹⁵N) in [²H₆]DMSO) or CDCl₃; coupling constants are in Hz. ¹⁵N NMR data reported are raw data and do not refer to internal or external standards. Purity was assessed using LC/MS. The LC/MS system consisted of an Agilent Technologies 1200 series LC connected to a 6110 Single Quadrupole MS with ESI source. Merck silica gel 60 (40-60 μm) was used for flash column chromatography.

### Synthesis 1

### tert-Butyl (8-carbamoyl-4-oxoimidazo[5,1-d][1,2,3,5]tetrazin-3(4H)-yl)methylcarbamate

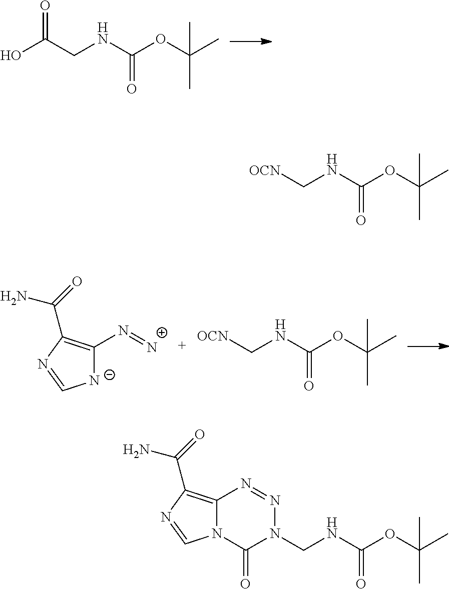

Ethyl chloroformate (573 μL, 5.99 mmol, 1.05 equ.) followed by triethylamine (556 μL, 5.99 mmol, 1.05 eq.) were added dropwise to a stirred solution of N-(tert-butoxycarbonyl) glycine in THF (20 mL) at 0° C. The mixture was stirred 45 minutes before the addition of an aqueous solution (5 mL) of sodium azide (557 mg, 8.57 mmol, 1.5 eq.) at 0° C. The resulting mixture was stirred at 0° C. for 1 h and was then diluted with water. The crude acyl azide was extracted four times with toluene and the combined organic extracts were successively washed with a saturated solution of sodium bicarbonate (twice), 1M HCl and water. The solution of acyl azide in toluene was dried over MgSO₄ at 0° C. and was then heated slowly with stirring until nitrogen gas evolution was observed, which occurred at 58° C. (oil bath temperature). The temperature of the oil bath was maintained at 63° C. for 1 h30 and was then increased slowly to 70° C. The solution was stirred at this temperature for 30 minutes and was concentrated under reduced pressure to give 1.1 g of tert-butyl isocyanatomethylcarbamate as a 1:1.42 mixture with toluene (ratio determined by ¹H NMR, 3.63 mmol of isocyanate, 64% yield). The isocyanate was used without further purification in the next step. IR (λₘₐₓ, cm⁻¹): 3308.0-3371.7 (w (br)), 2980.1 (w), 2245.2 (s), 1699.3 (s (br)), 1494.9 (s), 1367.6 (s), 1246.1 (s), 1153.5 (s), 947.1 (s), 846.8 (s), 729.1 (s). ¹H NMR (CDCl₃): 5.20 (s (br), 1H, NH), 4.53 (s (br), 2H, CH2), 1.36 (s, 9H, tBu).

1.02 g of the crude isocyanate (3.37 mmol, 1.32 eq.) was added dropwise in the dark under nitrogen to a stirred suspension of diazoimidazole carboxamide (350 mg, 2.55 mmol) in dry DMSO (4 mL) and the mixture was stirred overnight. The resulting solution was poured into ice and the precipitate was filtered, washed successively with water, ethyl acetate and diethyl ether to give 469 mg of the title compound as a pale pink solid (60% yield). ¹H NMR (DMSO d₆): 8.84 (s, 1H, CH), 8.03 (s (br), 1H, NH), 7.81 (s, 1H, CONH2), 7.69 (s, 1H, CONH2), 5.49 (d, 2H, J=6.3 Hz, CH2), 1.39 (s, 9H, tBu). LCMS: 94% pure, m/z (ES⁺): 332.0 (M+Na⁺)

### Synthesis 2

### NorTemozolomide Synthesis

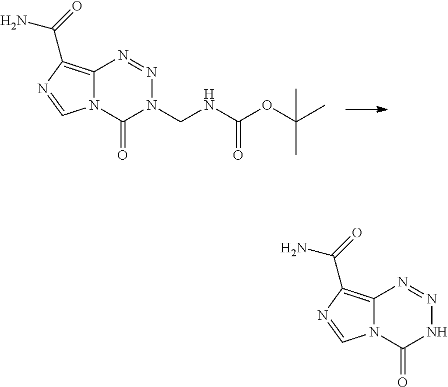 [SMILES: CC(C)(C)OC(=O)NCn1nnc2c(C(N)=O)ncn2c1=O ; NC(=O)c1ncn2c(=O)[nH]nnc12]

A suspension of tert-butyl (8-carbamoyl-4-oxoimidazo[5,1-d][1,2,3,5]tetrazin-3(4H)-yl)methylcarbamate (2.93 g, 9.47 mmol) in 3N HCl (130 mL) was stirred at room temperature overnight and was then kept at 4° C. for 4 hours. The precipitate was filtered and washed successively with water, ethyl acetate and diethyl ether to give the title compound (1.68 g, 98% yield) as a pale pink solid.

¹H NMR (DMSO d₆): 14.95 (s, 1H, NH), 8.77 (s, 1H, CH), 7.75 (s, 1H, CONH2), 7.65 (s, 1H, CONH2).

¹³C NMR (DMSO d₆): 162.1, 139.5, 135.0, 130.8, 129.0.

IR (λₘₐₓ, cm⁻¹): 3543.4 (w), 3446.91 (w), 3132.5 (m), 2704.3-3257.9 (m (br)), 1749.5 (s), 1672.3 (s), 1633.8 (s), 1601.0 (s), 1479.5 (m), 1442.8 (m), 1410.0 (m), 1242.2 (m), 1222.9 (s), 1151.5 (m), 1006.9 (m), 939.4 (m), 833.3 (m), 727.2 (s), 700.2 (s), 682.8 (s), 634.6 (s).

¹⁵N NMR (400 MHz; DMSO D₆): δ96.1 (s, 1N), 345.2 (s, 1N), 273.0-273.3 (d, J=11.3 Hz, 1N), 200.0 (s, 1N), 181.2-181.0 (d, J=7.9 Hz, 1N), 105.4 (s, 1N).

### Synthesis 3

### Temozolomide from NorTemozolomide

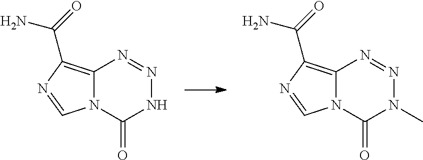

A 60% dispersion of sodium hydride in mineral oil (16.3 mg, 0.408 mmol, 1.05 eq.) was added in one portion to a slurry of 4-oxo-3,4-dihydroimidazo[5,1-d][1,2,3,5]tetrazine-8-carboxamide (70 mg, 0.389 mmol) in dry DMF (4.5 mL) at 0° C. The resulting suspension was stirred 3 minutes before the addition of methyl iodide (48 μL, 0.778 mmol, 2 eq.). The mixture became rapidly a green solution and was stirred at 0° C. for 30 minutes and then at room temperature for 2 h. The mixture was concentrated to dryness under high vacuum and the crude product was absorbed on silica. The crude product was purified by flash chromatography using DCM:MeOH 95:5 as eluant and two fractions were collected. The first fraction (46 mg, 61% yield) corresponded to the title product. ¹FI NMR (DMSO d₆): 8.82 (s, 1H, CH), 7.80 (s, 1H, CONH2), 7.67 (s, 1H, CONH2), 3.86 (s, 3H, CH3). The ¹H NMR data were consistent with the data of an original sample of temozolomide, as shown by an NMR spectrum of a mixture of temozolomide and the product obtained above. The second fraction (18 mg) was identified as a 1:0.70 mixture of the title compound and one of its regioisomers.

### Synthesis 4

### Derivatisation of NorTemozolomide

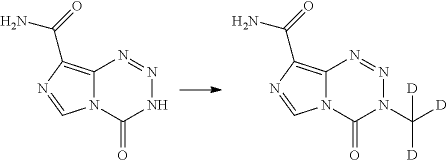 [SMILES: NC(=O)c1ncn2c(=O)[nH]nnc12 ; [2H]C([2H])([2H])n1nnc2c(C(N)=O)ncn2c1=O]

Nortemozolomide (0.65 g, 0.361 mmol) was stirred in anhydrous DMF (4 ml) at 0° C. then NaH (60% mineral oil, 0.016 g, 0.39 mmol) was added and the reaction was stirred for 5 mins at this temperature. CD₃I (45 uL. 0.163 g, 0.722 mmol) was then added and the reaction stirred for 30 mins at 0° C. then at RT overnight. The resulting black solution was then concentrated in vacuo to yield a black residue. This was purified by flash chromatography (silica gel, gradient elution, CH₂Cl₂ (100%) to CH₂Cl₂: MeOH, 10:1) to yield the title compound as a cream coloured solid (0.053 g, 75% yield). ¹H NMR (DMSO d₆): 8.82 (s, 1H, CH), 7.78 (s, 1H, CONH2), 7.66 (s, 1H, CONH2)

### Synthesis 5

### Derivatisation of NorTemozolomide

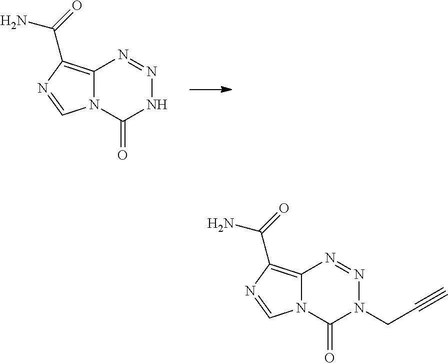 [SMILES: NC(=O)c1ncn2c(=O)[nH]nnc12 ; C#CCn1nnc2c(C(N)=O)ncn2c1=O]

Nortemozolomide (0.100 g, 0.550 mmol) was stirred in anhydrous DMF (5 ml) at 0° C. then NaH (60% mineral oil, 0.022 g, 0.550 mmol) was added and the reaction was stirred for 5 mins at this temperature. Propargyl bromide (80%, 0.163 g, 1.38 mmol) was then added and the reaction stirred for 30 mins at 0° C. then at RT overnight. The resulting black solution was then concentrated in vacuo to yield a black residue. This was purified by flash chromatography (silica gel, gradient elution, DCM (100%) to DCM: MeOH, 10:1) to yield 4-oxo-3-(prop-2-ynyl)-3,4-dihydroimidazo[5,1-d][1,2,3,5]tetrazine-8-carboxamide as a yellow/orange residue. This was sonicated in Et₂O and air dried to yield the title compound as a brown solid (0.038 g, 0.174 mmol, 32%). ¹H NMR (DMSO d₆): 8.86 (s, 1H, CH), 7.83 (s, 1H, CONH2), 7.71 (s, 1H, CONH2), 5.13 (d, 2H, CH2, J=2.5 Hz), 3.53 (t, 1H, CH, J=2.5 Hz).

### Synthesis 6

### Derivatisation of NorTemozolomide

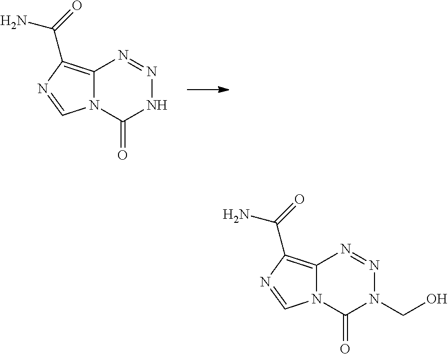 [SMILES: NC(=O)c1ncn2c(=O)[nH]nnc12 ; NC(=O)c1ncn2c(=O)n(CO)nnc12]

A slurry of 4-oxo-3,4-dihydroimidazo[5,1-d][1,2,3,5]tetrazine-8-carboxamide (50 mg, 0.277 mmol) in 1 mL of 37% aqueous solution of formaldehyde was stirred at room temperature for 72 hrs. After this time the suspension was filtered. The filtercake was washed with water (2 ml), EtOAc (2 mL) and finally Et₂O (2 mL) to give a pale pink solid which corresponded to the title compound (37 mg, 58% yield). ¹H NMR (DMSO d₆): 8.86 (s, 1H, CH), 7.82 (s, 1H, CONH2), 7.70 (s, 1H, CONH2), 7.22 (t, 1H, OH, J=7.7 Hz), 5.62 (d, 2H, CH2, J=7.7 Hz).

m/z (ES⁺): 232.9 (M+Na⁺), 210.90 (MH⁺), 203.10 (M-HCHO+Na⁺), 181.1 (MH⁺-HCHO).

### Synthesis 7

### Derivatisation of NorTemozolomide

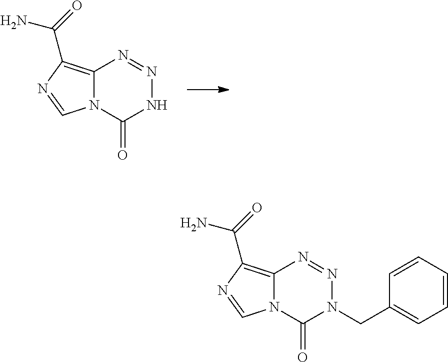 [SMILES: NC(=O)c1ncn2c(=O)[nH]nnc12 ; NC(=O)c1ncn2c(=O)n(Cc3ccccc3)nnc12]

To a cooled slurry of 4-oxo-3,4-dihydroimidazo[5,1-d][1,2,3,5]tetrazine-8-carboxamide (50 mg, 0.277 mmol, 1 eq.), polymer supported triphenylphosphine (371 mg, 3 mmol/g loading, 1.1 mmol 4 eq.) and benzyl alcohol (106 ul, 1.1 mmol, 4 eq.) in DMF (1 mL) was added diisopropylazodicarboxylate (210 uL, 1.1 mmol, 4 eq.). The mixture was stirred for 48 hrs before being diluted with MeCN (2 ml) and filtered through a thin Celite™ pad. The filtrate was concentrated in vacuo and absorbed onto silica before being purified by flash column chromatography (SiO₂) using CH₂Cl₂:MeOH (98:2 going to 95:5) to give the title compound as a white solid (19 mg, 25%). ¹H NMR (DMSO d₆): 8.83 (s, 1H, CH), 7.80 (s, 1H, CONH2), 7.68 (s, 1H, CONH2), 7.39-7.43 (m, 2H, ArCH), 7.31-7.39 (m, 3H, ArCH), 5.51 (s, 2H, CH2),

LCMS: 100% pure at 3.88 min., m/z (ES⁺): 293.1 (M+Na⁺), 271.1 (MH⁺)

### Synthesis 8

### Derivatisation of NorTemozolomide

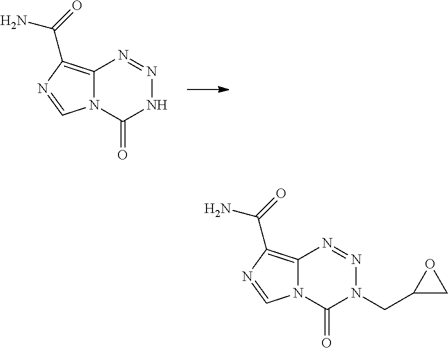 [SMILES: NC(=O)c1ncn2c(=O)[nH]nnc12 ; NC(=O)c1ncn2c(=O)n(CC3CO3)nnc12]

To a cooled slurry of 4-oxo-3,4-dihydroimidazo[5,1-d][1,2,3,5]tetrazine-8-carboxamide (100 mg, 0.55 mmol, 1 eq.), polymer supported triphenylphosphine (742 mg, 3 mmol/g loading, 2.2 mmol 4 eq.) and glycidol (150 ul, 2.2 mmol, 4 eq.) in DMF (2 mL) was added diisopropylazodicarboxylate (210 uL, 1.1 mmol, 4 eq.). The mixture was stirred for 48 hrs before being diluted with MeCN (2 ml) and filtered through a thin Celite™ pad. The filtrate was concentrated in vacuo and absorbed onto silica before being purified by flash column chromatography (SiO₂) using CH₂Cl₂:MeOH (98:2) to give a yellow residue which was then washed with EtOAc (2*5 ml) & Et2O (5 ml) to give the title compound as a pale orange solid (22 mg, 17% yield). ¹H NMR (DMSO d₆): 8.85 (s, 1H, CH), 7.81 (s, 1H, CONH2), 7.69 (s, 1H, CONH2), 4.58 (dd, 1H, CH2, J1=14.8 Hz, J2=3.7 Hz), 4.38 (dd, 1H, CH2, J1=14.8, J2=5.5 Hz), 3.40 (m, 1H, CH), 2.84 (m, 1H, CH2), 2.74 (dd, 1H, CH2, J1=4.9 Hz, J2=2.6 Hz) m/z (ES⁺): 259.1 (M+Na⁺).

### Synthesis 9

### 3-(Methoxymethyl)-4-oxo-3,4-dihydroimidazo[5,1-d][1,2,3,5]tetrazine-8-carboxamide and N,3-bis(methoxymethyl)-4-oxo-3,4-dihydroimidazo[5,1-d][1,2,3,5]tetrazine-8-carboxamide

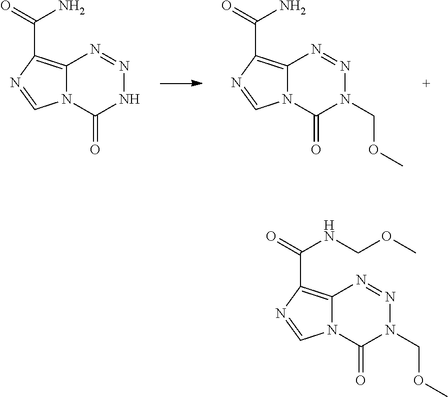 [SMILES: NC(=O)c1ncn2c(=O)[nH]nnc12 ; COCn1nnc2c(C(N)=O)ncn2c1=O ; "+" ; COCNC(=O)c1ncn2c(=O)n(COC)nnc12]

Nortemozolomide (0.100 g, 0.550 mmol) was stirred in anhydrous DMF (5 ml) at 0° C. then NaH (60% mineral oil, 0.022 g, 0.550 mmol) was added and the reaction was stirred for 5 mins at this temperature. Chloromethyl methyl ether (0.089 g, 1.100 mmol) was then added and the reaction stirred for 30 mins at 0° C. then at room temperature overnight. The resulting orange solution was concentrated in vacuo and purified by flash chromatography (silica gel, gradient elution, DCM (100%) to DCM: MeOH, 10:1) to yield N,3-bis(methoxymethyl)-4-oxo-3,4-dihydroimidazo[5,1-d][1,2,3,5]tetrazine-8-carboxamide (0.012 g, 0.045 mmol, 8%) and 3-(methoxymethyl)-4-oxo-3,4-dihydroimidazo[5,1-d][1,2,3,5]tetrazine-8-carboxamide as an approx 1:1 mixture with N,3-bis(methoxymethyl)-4-oxo-3,4-dihydroimidazo[5,1-d][1,2,3,5]tetrazine-8-carboxamide (0.045 g, 0.200 mmol).

N,3-bis(methoxymethyl)-4-oxo-3,4-dihydroimidazo[5,1-d][1,2,3,5]tetrazine-8-carboxamide; ∂_{H} (400 MHz; DMSO D₆) 9.28 (NH, t, J=8 Hz, 1H), 8.93 (CH, s, 1H), 5.63 (CH₂, s, 2H), 4.71 (CH₂, d, J=8 Hz, 2H), 3.42 (CH₃, s, 3H) and 3.26 (CH₃, s, 3H).

1. A compound of general formula (II) or (III), or a salt thereof: 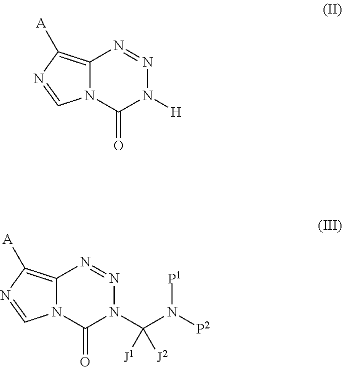
wherein
A is independently -A¹, -A², -A³, -A⁴, -A⁵, -A⁶, or -A⁷, wherein:
-A¹ is independently C₅₋₁₂heteroaryl, and is optionally substituted;
-A² is independently thioamido or substituted thioamido;
-A³ is independently imidamido, substituted imidamido, N-hydroxyimidamido, or substituted N-hydroxyimidamido;
-A⁴ is independently hydroxamic acid or hydroxamate;
-A⁵ is independently carboxamide or substituted carboxamide;
-A⁶ is independently aliphatic C₂₋₆alkenyl, and is optionally substituted; and
-A⁷ is independently carboxy or C₁₋₄alkyl-carboxylate;
J¹ and J² are each independently H or C₁₋₃ alkyl; and
P¹ and P² are each independently H or an amine protecting group
or P¹ and P² together form an amine protecting group.

2. A compound according to claim 1, wherein A is -A⁵.

3. A compound according to claim 2, wherein -A⁵ is —C(═O)NH₂.

4. A compound of formula (II) according to claim 1, which is nortemozolomide.

5. (canceled)

6. A compound of formula (III) according to claim 1, wherein one of P¹ and P² is —H and the other is an amine protecting group.

7. A compound of formula (III) according to claim 1, wherein the amine protecting group is an acid-cleavable protecting group.

8. A compound according to claim 7, wherein the amine protecting group is tert-butoxy carbonyl (Boc).

9. A compound of formula an) according to claim 1, wherein J¹ and J² are each independently selected from —H and -Me.

10. A compound of formula (III) according to claim 1, wherein J¹ and J² are each independently —H.

11-12. (canceled)

13. A method for the synthesis of a compound of formula (II) 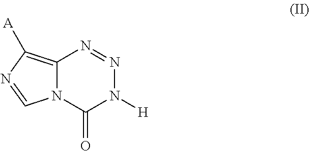
wherein
A is independently -A¹, -A², -A³, -A⁴, -A⁵, -A⁶, or -A⁷, wherein:
-A is independently C₅₋₁₂heteroaryl, and is optionally substituted;
-A² is independently thioamide or substituted thioamido;
-A³ is independently imidamido, substituted imidamido, N-hydroxyimidamido, or substituted N-hydroxyimidamido;
-A⁴ is independently hydroxamic acid or hydroxamate;
-A⁵ is independently carboxamide or substituted carboxamide;
-A⁶ is independently aliphatic C₂₋₆alkenyl, and is optionally substituted; and
-A⁷ is independently carboxy or C₁₋₄alkyl-carboxylate;
said method comprising deprotecting a compound of formula (III) 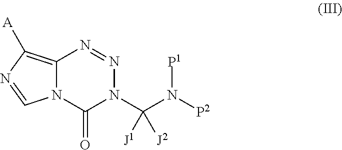
wherein A is as defined above;
J¹ and J² are each independently H or C₁₋₃ alkyl; and
P¹ and P² are each independently H or an amine protecting group
or P¹ and P² together form an amine protecting group.

14. A method according to claim 13, wherein deprotecting said compound of formula (III) comprises treatment with acid.

15. A method according to claim 13, wherein said compound of formula (III) is prepared by reaction of an isocyanate of general formula (IV): 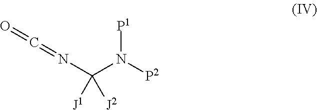
with a diazoimidazole compound of general formula (V): 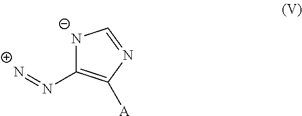
wherein A, J¹, J², P¹ and P² are as previously defined.

16. A method according to claim 15, wherein said isocyanate of formula (IV) is prepared from a protected amino acid of general formula (VI): 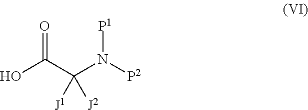
wherein J¹, J², P¹ and P² are as previously defined.

17. A method according to claim 16, wherein the protected amino acid of general formula (VI) is N-Boc-glycine.

18. (canceled)

19. A method for the synthesis of temozolomide or a temozolomide analogue, comprising reacting a compound of formula (II): 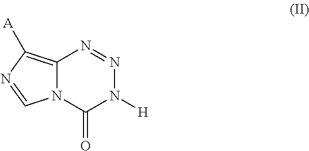
wherein
A is independently -A¹, -A², -A³, -A⁴, -A⁵, -A⁶, or -A⁷, wherein:
-A¹ is independently C₅₋₁₂heteroaryl, and is optionally substituted;
-A² is independently thioamido or substituted thioamido;
-A³ is independently imidamido, substituted imidamido, N-hydroxyimidamido, or substituted N-hydroxyimidamido;
-A⁴ is independently hydroxamic acid or hydroxamate;
-A⁵ is independently carboxamide or substituted carboxamide;
-A⁶ is independently aliphatic C₂₋₆ alkenyl, and is optionally substituted; and
-A⁷ is independently carboxy or C₁₋₄alkyl-carboxylate
with an electrophile.

20. A method according to claim 19, wherein said temozolomide or temozolomide analogue is a compound of general formula (I): 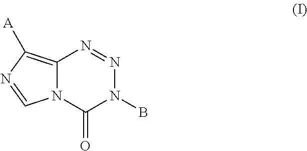
wherein A and B are as previously defined.

21. A method according to claim 19, wherein said electrophile is an alkylating agent.

22. A method according to claim 19, wherein said electrophile is an alkyl halide, an epoxide, an alkyl alcohol, an activated alkyl alcohol, an alkyl alkoxide or an aldehyde.

23. (canceled)

24. A method according to claim 19, wherein said electrophile is methyl iodide.

25. A method according to claim 19, wherein said electrophile is a compound of formula B—X, wherein B is as previously defined and X is a leaving group.

26. A method according to claim 19, wherein the compound of formula (II) is treated with a base prior to addition of the electrophile.

27. A method according to claim 19, wherein the compound of formula (II) is reacted with the electrophile via a Mitsunobu reaction.

28. A method according to claim 19, further comprising modification of the group at the 3-position of the reaction product to obtain a temozolomide analogue, and/or modification of the group at the 8-position of the reaction product to obtain a temozolomide analogue.

29. (canceled)
